SEQUENCE LISTING (33 sequences)

SEQ ID NO: 1 - protein, 9 aa, Artificial Sequence
  Description of Artificial SequenceNonapeptide derived from the human C-SKI Oncoprotein
ALAPPAQQK

SEQ ID NO: 2 - protein, 728 aa, Homo sapiens
MEAAAGGRGCFQPHPGLQKTLEQFHLSSMSSLGGPAAFSARWAQEAYKKESAKEAGAAAVPAPVPAATEPPPVLHLPAIQPPPPVLPGPFFMPSDRSTERCETVLEGETISCFVVGGEKRLCLPQILNSVLRDFSLQQINAVCDELHIYCSRCTADQLEILKVMGILPFSAPSCGLITKTDAERLCNALLYGGAYPPPCKKELAASLALGLELSERSVRVYHECFGKCKGLLVPELYSSPSAACIQCLDCRLMYPPHKFVVHSHKALENRTCHWGFDSANWRAYILLSQDYTGKEEQARLGRCLDDVKEKFDYGNKYKRRVPRVSSEPPASIRPKTDDTSSQSPAPSEKDKPSSWLRTLAGSSNKSLGCVHPRQRLSAFRPWSPAVSASEKELSPHLPALIRDSFYSYKSFETAVAPNVALAPPAQQKVVSSPPCAAAVSRAPEPLATCTQPRKRKLTVDTPGAPETLAPVAAPEEDKDSEAEVEVESREEFTSSLSSLSSPSFTSSSSAKDLGSPGARALPSAVPDAAAPADAPSGLEAELEHLRQALEGGLDTKEAKEKFLHEVVKMRVKQEEKLSAALQAKRSLHQELEFLRVAKKEKLREATEAKRNLRKEIERLRAENEKKMKEANESRLRLKRELEQARQARVCDKGCEAGRLRAKYSAQIEDLQVKLQHAEADREQLRADLLREREAREHLEKVVKELQEQLWPRARPEAAGSEGAAELEP

SEQ ID NO: 3 - DNA, 3511 bp, Homo sapiens
cggggcggcggcgggggccgggggggcccgggcgcgcgggagcgggagcggccgggggagccggagcgcaccatggaggcggcggcaggcggccgcggctgtttccagccgcacccggggctgcagaagacgctggagcagttccacctgagctccatgagctcgctgggcggcccggccgctttctcggcgcgctgggcgcaggaggcctacaagaaggagagcgccaaggaggcgggcgcggccgcggtgccggcgccggtgcccgcagccaccgagccgccgcccgtgctgcacctgcccgccatccagccgccgccgcccgtgctgcccgggcccttcttcatgccgtccgaccgctccaccgagcgctgcgagaccgtactggaaggcgagaccatctcgtgcttcgtggtgggaggcgagaagcgcctgtgtctgccgcagattctcaactcggtgctgcgcgacttctcgctgcagcagatcaacgcggtgtgcgacgagctccacatctactgctcgcgctgcacggccgaccagctggagatcctcaaagtcatgggcatcctgcccttctcggcgccctcgtgcgggctcatcaccaagacggacgccgagcgcctgtgcaacgcgctgctctacggcggcgcctacccgccgccctgcaagaaggagctggccgccagcctggcgctgggcctggagctcagcgagcgcagcgtccgcgtgtaccacgagtgcttcggcaagtgtaaggggctgctggtgcccgagctctacagcagcccgagcgccgcctgcatccagtgcctggactgccgcctcatgtacccgccgcacaagttcgtggtgcactcgcacaaggccctggagaaccggacctgccactggggcttcgactcggccaactggcgggcctacatcctgctgagccaggattacacgggcaaggaggagcaggcgcgcctcggccgctgcctggacgacgtgaaggagaaattcgactatggcaacaagtacaagcggcgggtgccccgggtctcctctgagcctccggcctccataagacccaaaacagatgacacctcttcccagtcccccgcgccttccgaaaaggacaagccgtccagctggctgcggaccttggccggctcttccaataagagcctgggctgtgttcaccctcgccagcgcctctctgctttccgaccctggtcccccgcagtgtcagcgagtgagaaagagctctccccacacctcccggccctcatccgagacagcttctactcctacaagagctttgagacagccgtggcgcccaacgtggccctcgcaccgccggcccagcagaaggttgtgagcagccctccgtgtgccgccgccgtctcccgggcccccgagcctctcgccacttgcacccagcctcggaagcggaagctgactgtggacaccccaggagccccagagacgctggcgcccgtggctgccccagaggaggacaaggactcggaggcggaggtggaagttgaaagcagggaggaattcacctcctccttgtcctcgctctcttccccgtcctttacctcatccagctccgccaaggacctgggctccccgggtgcgcgtgccctgccctcggccgtccctgatgctgcggcccctgccgacgcccccagtgggctggaggcggagctggagcacctgcggcaggcactggagggcggcctggacaccaaggaagccaaagagaagttcctgcatgaggtggtcaagatgcgcgtgaagcaggaggagaagctcagcgcagccctgcaggccaagcgcagcctccaccaggagctggagttcctacgcgtggccaagaaggagaagctgcgggaggccacggaggccaagcgtaacctgcggaaggagatcgagcgtctccgcgccgagaacgagaagaagatgaaagaggccaacgagtcacggctgcgcctgaagcgggagctggagcaggcgcggcaggcccgggtgtgcgacaagggctgcgaggcgggccgcctgcgcgccaagtactcggcccagatcgaagacctgcaggtgaagctgcagcacgcggaggcggaccgggagcagctgcgggccgacctgctgcgggagcgcgaggcccgggagcacctggagaaggtggtgaaggagctgcaggaacagctgtggccgcgggcccgccccgaggctgcgggcagcgagggcgctgcggagctggagccgtagattccgtgcctgccgccgcagcgccgccgacaacgcgggtgcaggggggcgcggctgggcggtgcagctccgcccggctccgcccctgcagcccacacagcacaacgtcttaccgtgcctattaccaagcgagtgtttgtaaccatgtagttttggaacccactgcaaaattttctactggccaagttcaagtgagtaagccgcgtcccccaactacagctggagacggggccagctcggcggcctgctggtcctctgcttgctggaacattctaacatttacacttttgttataagctatttaaaaccagtaaggagacttgaaattcagaaaatcaacacatttttaaatgactaacttctaaaagccccaacacatgacgccatctgaagacccgcaacggagtgggggtggcggccgccccaccctccccacccggggaagccatcacagctcatctgcccgcggctgcgtgaggacagcaggggtttttcttcagagtctattttttcagcgacaaggacccaggtcttcctgctgctgccagggagagcagggacagtgccgcgtgcgagatgagctcgaacactgcccgccttactgccgcctaccccgcccgccacgccgccgtcgatgccagcgctgtccccacgggtaccaggaagtgcagagccgcacaggagctgccccggagctgaggggacggtcttcggctcctctgcaccccgtgattctgcccacgctcctccaccacgaggcactgacctgcgtcgggtggtgaccgtggctggcggtcacgccctcagccctccgggcacacgtgccgcctgaccgggcgacccttttcagttcggcaaacgtcgctcccttcattttgggactgaggctgcagcattggaacaaaagagcattatttcaatttttctttctttttttttgttcgttcatttaaacgtatatttagaactgcactttgtccacaaccttcccttctctttctattccccagtgaactgaggtttttaccgactttatagagcagtcaaatccgaagtgctcgagtgcttagaaaccccctctggtgcttggttgaacaagggaatcacaaaaaaacgaaaatgcaaaaactgaacttcgggggtcgttctgtgccttccagcatcttgtacagcaaatcctgactcgtgtctttttacccccaagatatctgtcttcagtagcgactgaatctgccactctcagaataagttc

SEQ ID NO: 4 - protein, 9 aa, Artificial Sequence
  Description of Artificial SequenceNonapeptide derived from the human C-SKI oncoprotein.
ALFPPAQQK

SEQ ID NO: 5 - protein, 9 aa, Artificial Sequence
  Description of Artificial SequenceNonapeptide derived from the human C-SKI oncoprotein.
SSEPPASIR

SEQ ID NO: 6 - protein, 9 aa, Artificial Sequence
  Description of Artificial SequenceNonapeptide derived from the human C-SKI oncoprotein.
TADQLEILK

SEQ ID NO: 7 - protein, 9 aa, Artificial Sequence
  Description of Artificial SequenceNonapeptide derived from the human C-SKI oncoprotein.
ATEPPPVLH

SEQ ID NO: 8 - protein, 9 aa, Artificial Sequence
  Description of Artificial SequenceNonapeptide derived from the human C-SKI oncoprotein.
ELEFLRVAK

SEQ ID NO: 9 - protein, 9 aa, Artificial Sequence
  Description of Artificial SequenceNonapeptide derived from the human C-SKI oncoprotein.
ATEAKRNLR

SEQ ID NO: 10 - protein, 9 aa, Artificial Sequence
  Description of Artificial SequenceNonapeptide derived from the human C-SKI oncoprotein.
ALEGGLDTK

SEQ ID NO: 11 - protein, 9 aa, Artificial Sequence
  Description of Artificial SequenceNonapeptide derived from the human C-SKI oncoprotein.
RAENEKKMK

SEQ ID NO: 12 - protein, 9 aa, Artificial Sequence
  Description of Artificial SequenceNonapeptide derived from the human C-SKI oncoprotein.
ELEQARQAR

SEQ ID NO: 13 - protein, 9 aa, Artificial Sequence
  Description of Artificial SequenceNonapeptide derived from the human C-SKI oncoprotein.
EAEVEVESR

SEQ ID NO: 14 - protein, 9 aa, Artificial Sequence
  Description of Artificial SequenceNonapeptide derived from the human C-SKI oncoprotein.
LSERSVRVY

SEQ ID NO: 15 - protein, 9 aa, Artificial Sequence
  Description of Artificial SequenceNonapeptide derived from the human C-SKI oncoprotein.
LMYPPHKFV

SEQ ID NO: 16 - protein, 9 aa, Artificial Sequence
  Description of Artificial SequenceNonapeptide derived from the human C-SKI oncoprotein.
CLPQILNSV

SEQ ID NO: 17 - protein, 9 aa, Artificial Sequence
  Description of Artificial SequenceNonapeptide derived from the human C-SKI oncoprotein.
SLHQELEFL

SEQ ID NO: 18 - protein, 9 aa, Artificial Sequence
  Description of Artificial SequenceNonapeptide derived from the human C-SKI oncoprotein.
RLGRCLDDV

SEQ ID NO: 19 - protein, 9 aa, Artificial Sequence
  Description of Artificial SequenceNonapeptide derived from the human C-SKI oncoprotein.
YILLSQDYT

SEQ ID NO: 20 - protein, 9 aa, Artificial Sequence
  Description of Artificial SequenceNonapeptide derived from the human C-SKI oncoprotein.
VMGILPFSA

SEQ ID NO: 21 - protein, 9 aa, Artificial Sequence
  Description of Artificial SequenceNonapeptide derived from the human C-SKI oncoprotein.
ELSERSVRV

SEQ ID NO: 22 - protein, 9 aa, Artificial Sequence
  Description of Artificial SequenceNonapeptide derived from the human C-SKI oncoprotein.
FSLQQINAV

SEQ ID NO: 23 - protein, 9 aa, Artificial Sequence
  Description of Artificial SequenceNonapeptide derived from the human C-SKI oncoprotein.
KLTVDTPGA

SEQ ID NO: 24 - protein, 9 aa, Artificial Sequence
  Description of Artificial SequenceNonapeptide derived from the human C-SKI oncoprotein.
ALPSAVPDA

SEQ ID NO: 25 - protein, 9 aa, Artificial Sequence
  Description of Artificial SequenceNonapeptide derived from the human C-SKI oncoprotein.
KLSAALQAK

SEQ ID NO: 26 - protein, 9 aa, Artificial Sequence
  Description of Artificial SequenceNonapeptide derived from the human C-SKI oncoprotein.
KLREATEAK

SEQ ID NO: 27 - protein, 9 aa, Artificial Sequence
  Description of Artificial SequenceNonapeptide derived from the human C-SKI oncoprotein.
KMRVKQEEK

SEQ ID NO: 28 - protein, 9 aa, Artificial Sequence
  Description of Artificial SequenceNonapeptide derived from the human C-SKI oncoprotein.
ALEGGLDTK

SEQ ID NO: 29 - protein, 9 aa, Artificial Sequence
  Description of Artificial SequenceNonapeptide derived from the human C-SKI oncoprotein.
RVYHECFGK

SEQ ID NO: 30 - protein, 9 aa, Artificial Sequence
  Description of Artificial SequenceNonapeptide derived from the human C-SKI oncoprotein.
LLSQDYTGK

SEQ ID NO: 31 - protein, 9 aa, Artificial Sequence
  Description of Artificial SequenceNonapeptide derived from the human C-SKI oncoprotein.
RLRAENEKK

SEQ ID NO: 32 - protein, 9 aa, Artificial Sequence
  Description of Artificial SequenceNonapeptide derived from the human C-SKI oncoprotein.
KMKEANESR

SEQ ID NO: 33 - protein, 9 aa, Artificial Sequence
  Description of Artificial SequenceNonapeptide derived from the human C-SKI oncoprotein.
FLHEVVKMR

(19) United States
(12) Patent Application Publication
(10) Pub. No.: US 2002/0137683 A1
(43) Pub. Date: Sep. 26, 2002

(54) C-ski oncogene-derived peptides for prevention, treatment, and diagnosis of cancer

(76) Inventors: Kevin T. Hogan, Charlottesville, VA (US); Mark M. Ross, Charlottesville, VA (US)

Correspondence Address: CARELLA, BYRNE, BAIN, GILFILLAN, CECCHI,, STEWART & OLSTEIN, 6 BECKER FARM ROAD, ROSELAND, NJ, 07068, US

(21) Appl. No.: 09/753,831

(22) Filed: Jan. 3, 2001

Related U.S. Application Data
(60) Provisional application No. 60/174,296, filed on Jan. 3, 2000.

(51) Int. Cl. A61K 48/00; A61K 38/08; C07H 21/04; C07K 7/06

(52) U.S. Cl. 514/15; 514/44; 536/23.1; 530/328

(57) ABSTRACT

The present invention relates to compositions and methods for the prevention, treatment, and diagnosis of cancer, specifically malignant melanoma, colorectal carcinoma, ovarian carcinoma, lung carcinoma, and prostate carcinoma. The invention discloses peptides, polypeptides, and polynucleotides that can be used to stimulate a CTL response against cancer, and more specifically malignant melanoma, colorectal carcinoma, ovarian carcinoma, lung carcinoma, and prostate carcinoma.

This Application claims priority based on U.S. Provisional Application Serial No. 60/174296, filed Jan. 3, 2000, the disclosure of which is hereby incorporated by reference in its entirety.

### FIELD OF THE INVENTION

The present invention relates to immunogens comprising epitopic peptides derived from the c-ski oncoprotein, a product of the c-ski oncogene, and to uses of said immunogens in eliciting cytotoxic T lymphocyte responses both in vitro and in vivo, especially for the diagnosis, prevention and treatment of cancer.

### BACKGROUND OF THE INVENTION

The present invention relates to peptides and polypeptides that facilitate a cytotoxic T lymphocyte (CTL)-mediated immune response against melanoma and other cancers. The present invention also relates to nucleic acid molecules that encode for such peptides and polypeptides, and which can also be used to facilitate an immune response against melanoma and other cancers.

The mammalian immune system has evolved a variety of mechanisms to protect the host from cancerous cells. An important component of this response is mediated by cells referred to as T cells. CTLs are specialized T cells that function primarily by recognizing and killing cancerous cells or infected cells, but they can also function by secreting soluble molecules referred to as cytokines that can mediate a variety of effects on the immune system.

A variety of evidence suggest that immunotherapy designed to stimulate a tumor-specific CTL response would be effective in controlling cancer. For example, it has been shown that human CTLs recognize sarcomas (Slovin, S. F. et al., J. Immunol., 137:3042-3048, (1987)), renal cell carcinomas (Schendel, D. J. et al., J.Immunol., 151:4209-4220, (1993)), colorectal carcinomas (Jacob, L. et al., Int.J.Cancer, 71:325-332, (1997)), ovarian carcinomas (Ioannides, C. G. et al., J.Immunol., 146:1700-1707, (1991)) (Peoples, G. E. et al., Surgery, 114:227-234, (1993)), pancreatic carcinomas (Peiper, M. et al., Eur.J.Immunol., 27:1115-1123, (1997); Wolfel, T. et al., Int.J.Cancer, 54:636-644, (1993)), squamous tumors of the head and neck (Yasumura, S. et al., Cancer Res., 53:1461-1468, (1993)), and squamous carcinomas of the lung (Slingluff, C. L. Jr et al., Cancer Res., 54:2731-2737, (1994); Yoshino, I. et al., Cancer Res., 54:3387-3390, (1994)). The largest number of reports of human tumor-reactive CTLs, however, have concerned melanomas (Boon, T. et al., Ann.Rev.Immunol., 12:337-365, (1994)). The ability of tumor-specific CTLs to mediate tumor regression, in both human (Rosenberg, S. A. et al., N.Engl.J.Med., 319:1676-1680, (1988))and animal models (Celluzzi, C. M. et al., J.Exp.Med., 183:283-287, (1996); Mayordomo, J. I. et al., Nat.Med., 1:1297-1302, (1995); Zitvogel, L. et al., J.Exp.Med., 183:87-97, (1996)), suggests that methods directed at increasing CTL activity would likely have a beneficial effect with respect to tumor treatment.

In the United States, melanoma, or skin cancer, is a disease that is diagnosed in approximately 44,000 persons per year. Conventional therapy for the disease includes surgery, radiation therapy, and chemotherapy. In spite of these approaches to treatment, approximately 7,300 individuals die in the United States every year due to melanoma. Overall, the 5-year survival rate for the disease is 88%. The survival rate drops, however, in more advanced stages of the disease with only about 50% of Stage III patients, and 20-30% of Stage IV patients surviving past five years. In patients in which the melanoma has metastasized to distant sites, the 5-year survival dips to only 12%. Clearly, there is a population of melanoma patients that is in need of better treatment options. More recently, in an attempt to decrease the number of deaths attributed to melanoma, immunotherapy has been added to the arsenal of treatments used against the disease.

In order for CTLs to kill or secrete cytokines in response to a cancer cell, the CTL must first recognize that cell as being cancerous. This process involves the interaction of the T cell receptor, located on the surface of the CTL, with what is generically referred to as an MHC-peptide complex which is located on the surface of the cancerous cell. MHC (major histocompatibility-complex)-encoded molecules have been subdivided into two types, and are referred to as class I and class II MHC-encoded molecules.

In the human immune system, MHC molecules are referred to as human leukocyte antigens (HLA). Within the MHC, located on chromosome six, are three different genetic loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, and HLA-B8 are examples of different class I MHC molecules that can be expressed from these loci. The present disclosure involves peptides that are associated with the HLA-A1, -A2, and -A3 molecules, and with the gene and protein that gives rise to these peptides.

The peptides that associate with the MHC molecules can either be derived from proteins made within the cell, in which case they typically associate with class I MHC molecules (Rock, K. L. and Golde, U., Ann.Rev.Immunol., 17:739-779, (1999)); or they can be derived from proteins that are acquired from outside of the cell, in which case they typically associate with class II MHC molecules (Watts, C., Ann.Rev.Immunol., 15:821-850, (1997)). Peptides that evoke a cancer-specific CTL response most typically associate with class I MHC molecules. The peptides that associate with a class I MHC molecule are typically nine amino acids in length, but can vary from a minimum length of eight amino acids to a maximum of fourteen amino acids in length. A class I MHC molecule with its bound peptide, or a class II MHC molecule with its bound peptide, is referred to as an MHC-peptide complex.

The process by which intact proteins are degraded into peptides is referred to as antigen processing. Two major pathways of antigen processing occur within cells (Rock, K. L. and Golde, U., Ann.Rev.Immunol., 17:739-779, (1999); Watts, C., Ann.Rev.Immunol., 15:821-850, (1997)). One pathway, which is largely restricted to cells that are antigen presenting cells such as dendritic cells, macrophages, and B cells, degrades proteins that are typically phagocytosed or endocytosed into the cell. Peptides derived in this pathway typically bind to class II MHC molecules. A second pathway of antigen processing is present in essentially all cells of the body. This second pathway primarily degrades proteins that are made within the cells, and the peptides derived from this pathway primarily bind to class I MHC molecules. It is peptides from this second pathway of antigen processing that are referred to herein. Antigen processing by this latter pathway involves polypeptide synthesis and proteolysis in the cytoplasm. The peptides produced are then transported into the endoplasmic reticulum of the cell, associate with newly synthesized class I MHC molecules, and the resulting MHC-peptide complexes are then transported to the cell surface. Peptides derived from membrane and secreted proteins have also been identified. In some cases these peptides correspond to the signal sequence of the proteins that are cleaved from the protein by the signal peptidase. In other cases, it is thought that some fraction of the membrane and secreted proteins are transported from the endoplasmic reticulum into the cytoplasm where processing subsequently occurs.

Once bound to the class I MHC molecule and displayed on the surface of a cell, the peptides are recognized by antigen-specific receptors on CTL. Mere expression of the class I MHC molecule itself is insufficient to trigger the CTL to kill the target cell if the antigenic peptide is not bound to the class I MHC molecule. Several methods have been developed to identify the peptides recognized by CTL, each method relying on the ability of a CTL to recognize and kill only those cells expressing the appropriate class I MHC molecule with the peptide bound to it (Rosenberg, S. A., Immunity, 10:281-287, (1999)). Such peptides can be derived from a non-self source, such as a pathogen (for example, following the infection of a cell by a bacterium or a virus) or from a self-derived protein within a cell, such as a cancerous cell. Examples of sources of self-derived proteins in cancerous cells have been reviewed (Gilboa, E., Immunity, 11:263-270, (1999); Rosenberg, S. A., Immunity, 10:281-287, (1999)) and include: (i) mutated genes; (ii) aberrantly expressed genes such as an alternative open reading frame or through an intron-exon boundary; (iii) normal genes that are selectively expressed in only the tumor and the testis; and (iv) normal differentiation genes that are expressed in the tumor and the normal cellular counterpart.

Four different methodologies have typically been used for identifying the peptides that are recognized by CTL. These are: (i) the genetic method; (2) motif analysis; (3) SErological analysis of REcombinant cDNA expression libraries (SEREX™); and (iv) the analytical chemistry approach or the Direct Identification of Relevant Epitopes for Clinical Therapeutics (DIRECT™).

The genetic method is an approach in which progressively smaller subsets of cDNA libraries from tumor cells are transfected into cells that express the appropriate MHC molecule but not the tumor-specific epitope. The molecular clones encoding T cell epitopes are identified by their ability to reconstitute tumor specific T cell recognition of transfected cells. The exact T cell epitope is then identified by a combination of molecular subcloning and the use of synthetic peptides based on the predicted amino acid sequence. Such methods, however, are susceptible to inadvertent identification of cross-reacting peptides, and are not capable of identifying important post-translational modifications.

Motif analysis involves scanning a protein for peptides containing known class I MHC binding motifs, followed by synthesis and assay of the predicted peptides for their ability to be recognized by tumor-specific CTL. This approach requires prior knowledge of the protein from which the peptides are derived. This approach is also greatly hampered by the fact that not all of the predicted peptide epitopes are presented on the surface of a cell (Yewdell, J. W. and Bennink, J. R., Ann.Rev.Immunol., 17:51-88, (1999)), thus additional experimentation is required to determine which of the predicted epitopes is useful.

The SEREX™ approach relies on using antibodies in the serum of cancer patients to screen cDNA expression libraries for a clone that expresses a protein recognized by the antibody. This methodology presumes that an antibody response will necessarily have developed in the presence of a T cell response, and thus, the identified clone is a good candidate to encode a protein that can be recognized by T cells.

DIRECT™ involves a combination of cellular immunology and mass spectrometry. This approach involves the actual identification of CTL epitopes by sequencing the naturally occurring peptides associated with class I MHC molecules. In this approach, cells are first lysed in a detergent solution, the peptides associated with the class I MHC molecules are purified, and the peptides fractionated by high performance liquid chromatography (HPLC). The peptides are then used to reconstitute recognition by tumor-specific CTLs on a non-tumor cell expressing the appropriate MHC molecules. Sequencing is readily performed by tandem mass spectrometry (Henderson, R. A. et al., Proc.Natl.Acad.Sci.U.S.A, 90:10275-10279, (1993); Hogan, K. T. et al., Cancer Res., 58:5144-5150, (1998); Hunt, D. F. et al., Science, 255:1261-1263, (1992); Slingluff, C. L. Jr et al., J.Immunol., 150:2955-2963, (1993)). During the course of employing this approach for the identification of melanoma-derived peptides, it was discovered that a c-ski oncogene-derived peptide bound to HLA-A3, thus indicating for the first time that the c-ski oncoprotein is a potential immunotherapeutic target.

The c-ski oncogene product is a nuclear regulatory protein that is expressed at low levels in a variety of tissue types and may play a role in muscle development (Colmenares, C. and Stavnezer, E., Cancer Biology, 1:383-387, (1990)). It has also been shown to be over-expressed in melanoma cells relative to normal melanocytes (Fumagalli, S. et al., Melanoma Res., 3:23-27, (1993)). More recently, c-ski has been shown to regulate the TGF-α signaling pathway (Vogel, G., Science, 286:665-665, (1999)).

Immunization with melanoma-derived, class I MHC-encoded molecule associated peptides, or with a precursor polypeptide or protein that contains the peptide, or with a gene that encodes a polypeptide or protein containing the peptide, are forms of immunotherapy that can be employed in the treatment of melanoma. These forms of immunotherapy require that immunogens be identified so that they can be formulated into an appropriate vaccine. Although a variety of melanoma-derived antigens have been identified (Rosenberg, S. A., Immunity, 10:281-287, (1999)), not all of these are appropriate for broad-based immunotherapy as the expression of some peptides is limited to the tumor derived from a specific patient. Furthermore, the number of class I MHC molecules from which tumor-derived peptides have been discovered is largely restricted to HLA-A2. Thus, it would be useful to identify additional peptides that complex with class I MHC molecules other than HLA-A2. Such peptides would be particularly useful in the treatment of melanoma patients who do not express the HLA-A2 molecule. It is also particularly useful to identify antigenic peptides that are derived from different parent proteins, even if the derived peptides associate with the same class I MHC molecule. Because an active immune response can result in the outgrowth of tumor cells that have lost the expression of a particular precursor protein for a given antigenic peptide, it is advantageous to stimulate an immune response against peptides derived from more than one parent protein, as the chances of the tumor cell losing the expression of both proteins is the multiple of the chances of losing each of the individual proteins.

### SUMMARY OF THE INVENTION

The present invention provides compositions comprising immunogenic peptides and polypeptides, and polynucleotides that direct the synthesis of such peptides and polypeptides, whereby said peptides and polypeptides are capable of inducing a CTL response against cells expressing a protein comprising an epitope having the sequence of SEQ ID NO:1 that is presented in association with HLA-A3; SEQ ID NO:5-14 presented in association with HLA-A1; SEQ ID NO:15-24 presented in association with HLA-A2; or SEQ ID NO:4 and 25-33 presented in association with HLA-A3. The cells are usually tumor cells, such as melanoma, colorectal carcinoma, ovarian carcinoma, lung carcinoma, or prostate carcinoma, expressing a protein of the c-ski oncogene (SEQ ID NO:3). The immunogenic polypeptides, and polynucleotides that direct the synthesis of such polypeptides, can also be used to induce a CTL response against other c-ski (SEQ ID NO:2) -derived peptides that do not comprise SEQ ID NO:1 or 5-33. Such peptides may associate with HLA-A1, -A2, -A3 or other class I MHC molecules, and thus would find use in inducing a CTL response against tumor cells expressing the c-ski oncoprotein, regardless of the cells expression of HLA-A3.

The peptides of the invention are usually 9 residues in length, and have a sequence that comprises SEQ ID NO:1, 4-33. The polypeptides of the invention can be of any length as long as part of their sequence comprises at least one peptide of SEQ ID NO:1, 4-33 when it is desired to induce a CTL response against a peptide of the corresponding sequence. Said polypeptides could correspond to the full-length c-ski protein (SEQ ID NO:2), to a fragment of the c-ski protein that contains a peptide corresponding to at least one of SEQ ID NO:1, 5-33, or they could be artificially synthesized polypeptides, a portion of which comprises a peptide of at least one of SEQ ID NO:1 or 4-33.

Thus, in one embodiment, the present invention relates to an isolated peptide of at least 8 amino acid residues in length and having an amino acid sequence at least 85% identical to the sequence selected from the group consisting of SEQ ID NOS:1, 4 to 33. In another embodiment, the present invention is directed to an isolated peptide of no more than about 14 amino acids in length comprising a sequence selected from the group consisting of SEQ ID NOS:1, 4 to 33. Specific embodiments of the invention disclosed herein relates to an isolated nonapeptide having an amino acid sequence selected from the group consisting of SEQ ID NOS:1, 4 to 33 and an isolated nonapeptide having a sequence differing by no more than 1 amino acid from a sequence selected from the group consisting of SEQ ID NOS:1, 4 to 33 and especially wherein in the latter embodiment, the amino acid difference is a substitution of one conservative amino acid for another.

The polynucleotides of the invention comprise the gene coding for c-ski (SEQ ID NO:3), a gene fragment of the gene encoding c-ski that minimally encodes for a peptide comprising at least one of SEQ ID NO:1 or 5-33, or a synthetic gene fragment that minimally encodes for a peptide comprising SEQ ID:1 or4-33.

The methods of the invention comprise contacting a lymphocyte with an immunogenic peptide under conditions that induce a CTL response against a tumor cell, and more specifically against a melanoma cell. The methods may involve contacting the CTL with the immunogenic peptide in vivo, in which case the peptides, polypeptides, and polynucleotides of the invention are used as vaccines, and will be delivered as a pharmaceutical composition comprising a pharmaceutically acceptable carrier and the immunogen, typically along with an adjuvant or one or more cytokines. Alternatively, the immunogens can be used to induce a CTL response in vitro. The generated CTL can then be introduced into a patient with cancer, more specifically melanoma, colorectal carcinoma, ovarian carcinoma, lung carcinoma, or prostate carcinoma. Alternatively, the ability to generate CTL in vitro can serve as a diagnostic for melanoma, colorectal carcinoma, ovarian carcinoma, lung carcinoma, or prostate carcinoma.

### BRIEF DESCRIPTION OF THE DRAWINGS

FIG. 1. Mass spectrometric analysis of the m/z 462.62²⁺candidate peptide and its synthetic counterpart. (A) The predicted masses of the type b and y fragment ions are shown above and below, respectively, as well as the deduced amino acid sequence that was obtained from both by interpretation of the unknown peptide mass spectrum (B) and is identical to that of the synthetic peptide (C) that corresponds to SEQ ID NO:1.

FIG. 2. Representative gel showing the expression of a c-ski 370 bp RT-PCR product in 16 different cell lines. 30 cycles of amplification were used for the top panel and 35 cycles for the bottom panel. Differing band intensities with 30 cycles indicates different levels of gene expression. The following melanoma lines were tested: DM92 (Lane 1), DM122 (Lane 2), DM154 (Lane 3), DM342 (Lane 4), DM366 (Lane 5), DM390 (Lane 6), DM391 (Lane 7), DM472 (Lane 8), DM607 (Lane 9), DM661B (Lane 10), SK-Mel2 (Lane 11), SK-Mel24 (Lane 12), VMM12 (Lane 13), VMM15 (Lane 14), and VMM18 (Lane 16). JY, a B-lymphoblastoid cell line was also tested (Lane 15), and Lane 17 was a no DNA control.

FIG. 3. Expression of the c-ski 370 bp RT-PCR product in a variety of normal, adult human tissues. 30 cycles of amplification were used for the top panel and 35 cycles for the bottom panel. The amplification mixture in each lane was derived from: skeletal muscle (Lane 1), liver (Lane 2), pancreas (Lane 3), placenta (Lane 4), heart (Lane 5), lung (Lane 6), kidney (Lane 7), brain (Lane 8), DM661B, a melanoma cell line (Lane 9), and a no DNA control (Lane 10).

FIG. 4. Gel showing the expression of c-ski RT-PCR product (Panels A and B) or the control G3PDH product (Panel C) in colorectal, ovarian, lung, and prostate carcinoma lines. 30 cycles of amplification was used for Panel A, 35 cycles of amplification was used for Panel B, and 20 cycles of amplification were used for Panel C. The amplification mixture in each lane is shown on the figure.

### DEFINITIONS

As used herein and except as noted otherwise, all terms are defined as given below.

The term “peptide” is used herein to designate a series of amino acid residues, connected one to the other typically by peptide bonds between the alpha-amino and carbonyl groups of the adjacent amino acids. The peptides are typically 9 amino acids in length, but can be as short as 8 amino acids in length, and as long as 14 amino acids in length.

The term “oligopeptide” is used herein to designate a series of amino acid residues, connected one to the other typically by peptide bonds between the alpha-amino and carbonyl groups of the adjacent amino acids. The length of the oligopeptide is not critical to the invention as long as the correct epitope or epitopes are maintained. The oligopeptides are typically less than about 30 amino acid residues in length, and greater than about 14 amino acids in length.

The term “polypeptide” designates a series of amino acid residues, connected one to the other typically by peptide bonds between the alpha-amino and carbonyl groups of the adjacent amino acids. The length of the polypeptide is not critical to the invention as long as the correct epitopes are maintained. In contrast to the terms peptide or oligopeptide, the term polypeptide is meant to refer to protein molecules of longer than about 30 residues in length.

A peptide, oligopeptide, protein, or polynucleotide coding for such a molecule is “immunogenic” (and thus an “immunogen” within the present invention) if it is capable of inducing an immune response. In the case of the present invention, immunogenicity is more specifically defined as the ability to induce a CTL-mediated response. Thus, an “immunogen” would be a molecule that is capable of inducing an immune response, and in the case of the present invention, a molecule capable of inducing a CTL response.

A T cell “epitope” is a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

The term “nucleotide sequence” refers to a heteropolymer of deoxyribonucleotides. The nucleotide sequence encoding for a particular peptide, oligopeptide, or polypeptide may be naturally occurring or they may be synthetically constructed. Generally, DNA segments encoding the peptides, polypeptides, and proteins of this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The term “DNA segment” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, by using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

The term “primer” means a short nucleic acid sequence that is paired with one strand of DNA and provides a free 3'OH end at which a DNA polymerase starts synthesis of a deoxyribonucleotide chain.

The term “promoter” means a region of DNA involved in binding of RNA polymerase to initiate transcription.

The term “open reading frame (ORF)” means a series of triplets coding for amino acids without any termination codons and is a sequence (potentially) translatable into protein.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The polynucleotides, and recombinant or immunogenic polypeptides, disclosed in accordance with the present invention may also be in “purified” form. The term “purified” does not require absolute purity; rather, it is intended as a relative definition, and can include preparations that are highly purified or preparations that are only partially purified, as those terms are understood by those of skill in the relevant art. For example, individual clones isolated from a cDNA library have been conventionally purified to electrophoretic homogeneity. Purification of starting material or natural material to at least one order of magnitude, preferably two or three orders, and more preferably four or five orders of magnitude is expressly contemplated. Furthermore, the claimed polypeptide which has a purity of preferably 0.001%, or at least 0.01% or 0.1%; and even desirably 1% by weight or greater is expressly contemplated.

The nucleic acids and polypeptide expression products disclosed according to the present invention, as well as expression vectors containing such nucleic acids and/or such polypeptides, may be in “enriched form.” As used herein, the term “enriched” means that the concentration of the material is at least about 2, 5, 10, 100, or 1000 times its natural concentration (for example), advantageously 0.01%, by weight, preferably at least about 0.1% by weight. Enriched preparations of about 0.5%, 1%, 5%, 10%, and 20% by weight are also contemplated. The sequences, constructs, vectors, clones, and other materials comprising the present invention can advantageously be in enriched or isolated form.

The term “active fragment” means a fragment that generates an immune response (i.e., has immunogenic activity) when administered, alone or optionally with a suitable adjuvant, to an animal, such as a mammal, for example, a rabbit or a mouse, and also including a human, such immune response taking the form of stimulating a CTL response within the recipient animal, such as a human. Alternatively, the “active fragment” may also be used to induce a CTL response in vitro.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. This means that any such fragment will necessarily contain as part of its amino acid sequence a segment, fragment or portion, that is substantially identical, if not exactly identical, to a sequence of SEQ ID NOs:1, 5-33. When used in relation to polynucleotides, such terms refer to the products produced by treatment of said polynucleotides with any of the common endonucleases.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100 [1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated Percent Identity is less than the specified Percent Identity.

### DETAILED DESCRIPTION OF THE INVENTION

The present invention relates generally to immunogens and immunogenic compositions, and methods of use therefor, for the prevention, treatment, and diagnosis of cancer, specifically malignant melanoma, colorectal carcinoma, ovarian carcinoma, lung carcinoma, and prostate carcinoma. The invention discloses peptides, polypeptides, and polynucleotides that can be used to stimulate a CTL response against cancer, and more specifically malignant melanoma, colorectal carcinoma, ovarian carcinoma, lung carcinoma, and prostate carcinoma.

The present invention relates to isolated peptides, especially epitopic peptides, of at least about 8 amino acids in length, preferably about 9 amino acids in length (i.e., nonapeptides), and no longer than about 14 amino acids in length, certainly no larger than about 15 amino acids in length, and having an amino acid sequence at least about 85% identical to an amino acid sequence selected from the group consisting of the amino acid sequences of SEQ ID NOS: 1 and 4 through 33. The sequences of isolated peptides of the present invention also can differ from the sequences of SEQ ID NO:1 by no more than 1 amino acid residue, preferably a conservative amino acid residue. Said isolated peptides are commonly immunogens, or at least can have immunogenic activity, possibly requiring a larger carrier molecule to facilitate such activity, or said peptides may have immunogenic activity when part of a larger structure, such as a polypeptide, other than the c-ski protein itself. Such peptides may also have immunogenic activity when part of a composition containing one or more of said epitopic peptides, which may be present in any combination and with each such peptide being present in one or more copies.

Thus, the epitopic, or immunogenic, peptides (SEQ ID NOs:1, 4-33) of the present invention may be natural peptides, or they may be produced by synthetic or recombinant methodologies that are well known and clear to the skilled artisan (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York). Besides peptides comprising SEQ ID NOs:1, 4-33, the immunogens of the present invention may also comprise one or more other immunogenic peptides that are known to be associated with cancer, and more specifically with melanoma, colorectal carcinoma, ovarian carcinoma, lung carcinoma, or prostate carcinoma, and which may stimulate a CTL response whereby the immunogenic peptides associate with HLA-A3 or another class I MHC molecule. Said immunogenic peptides could be in the form of a pool of peptides, or a composition comprising one or more of the immunogenic peptides, each present in almost any ratio of concentration relative to the others, including pools or compositions containing copies of one of the immunogenic peptides, or possibly 2, or 3, or more, each present in a concentration either related or unrelated to that of each, or some, or all of the other peptides. Such compositions can, therefore, be homogeneous or heterogeneous with respect to the individual immunogenic peptide components present therein, having only one or more than one of such peptides. For example, an isolated peptide of the present invention can have the sequence of SEQ ID NO:4.

The immunogenic peptides of the present invention may likewise be represented within an oligopeptide or polypeptide that comprises a peptide of at least one of SEQ ID NO:1, 4-33. The oligopeptides and polypeptides may be derived by fractionation of the naturally occurring c-ski protein by methods such as protease treatment, or they may be produced by recombinant or synthetic methodologies that are well known and clear to the skilled artisan (Ausubel, F. M. et al, Current Protocols in Molecular Biology, 1999, John Wiley & Sons, Inc., New York; Coligan, J. E. et al, Current Protocols in Protein Science, 1999, John Wiley & Sons, Inc., New York; Molecular Cloning: A Laboratory Manual, 1989, Cold Spring Harbor Laboratory Press, Cold Spring Harbor). The polypeptide may comprise: the entire c-ski oncogene-derived protein (SEQ ID NO:2); fragments of the c-ski oncogene-derived protein that necessarily comprise at least one of SEQ ID NOs:1, 5-33; a recombinant or synthetic polypeptide that necessarily comprises at least one of SEQ ID NOs:1, 5-33; any other sequences or combination of sequences derived from the c-ski protein such as a sequence comprising SEQ ID NO:4. Thus, oligopeptides and polypeptides of the present invention may have one, two, three, or more such immunogenic peptides within the amino acid sequence of said oligopeptides and polypeptides, and said immunogenic peptides, or epitopes, may be the same or may be different, or may have any number of such epitopes wherein some of them are identical to each other (in amino acid sequence) while others within the same polypeptide sequence are different from each other and said epitopes may occur in any order within said immunogenic polypeptide sequence. The location, arrangement, and ordering of the immunogenic peptides within the sequence of an immunogenic oligopeptide or polypeptide of the present invention will probably, although not necessarily, affect the ability of the various epitopes to be processed from the oligopeptide or polypeptide and to consequently be available to bind to the HLA-A3 or other class I MHC molecules. Thus, the location, arrangement, and ordering of these immunogenic peptides affords the user, either researcher or clinician, with the opportunity to regulate such interaction, or interactions, on the basis of the sequence of the immunogenic oligopeptide or polypeptide generated according to the methods disclosed herein.

Thus, in one embodiment, the present invention relates to an isolated peptide of at least 8 amino acid residues in length and having an amino acid sequence at least 85% identical to the sequence selected from the group consisting of SEQ ID NOS:1, 4 to 33. In another embodiment, the present invention is directed to an isolated peptide of no more than about 14 amino acids in length comprising a sequence selected from the group consisting of SEQ ID NOS:1, 4 to 33. Specific embodiments of the invention disclosed herein relates to an isolated nonapeptide having an amino acid sequence selected from the group consisting of SEQ ID NOS:1, 4 to 33 and an isolated nonapeptide having a sequence differing by no more than 1 amino acid from a sequence selected from the group consisting of SEQ ID NOS:1, 4 to 33 and especially wherein in the latter embodiment, the amino acid difference is a substitution of one conservative amino acid for another.

Where the immunogen comprises two or more immunogenic epitopes, or epitopic peptides, they may be linked directly together, or through a spacer or linker, to form a larger structure, such as an oligopeptide, or polypeptide, or some other polymeric structure. The epitopic peptides may therefore be linked by any and all means that can be devised by the chemist so long as the immunogenic activity of the overall structure or complex is maintained or, at least, not reduced below a level useful for the methods of the invention (i.e., especially where said immunogenic activity comprises being capable of eliciting a CTL response). Likewise, the immunogenic peptide may be linked directly to, or through a spacer or linker to: an immunogenic carrier such as serum albumin, tetanus toxoid, keyhole limpet hemocyanin, dextran, or a recombinant virus particle; an immunogenic peptide known to stimulate a T helper cell type immune response; a cytokine such as interferon gamma or GMCSF; a targeting agent such as an antibody or receptor ligand; a stabilizing agent such as a lipid; or a conjugate of a plurality of epitopes to a branched lysine core structure, such as the so-called “multiple antigenic peptide” described in (Posneft, D. N. et al., J.Biol.Chem., 263:1719-1725, (1988)); a compound such as polyethylene glycol to increase the half life of the peptide; or additional amino acids such as a leader or secretory sequence, or a sequence employed for the purification of the mature sequence. Spacers and linkers are typically comprised of relatively small, neutral molecules, such as amino acids and which are substantially uncharged under physiological conditions. Such spacers are typically selected from the group of nonpolar or neutral polar amino acids, such as glycine, alanine, serine and other similar amino acids. Such optional spacers or linkers need not be comprised of the same residues and thus may be either homo- or hetero-oligomers. When present, such linkers will commonly be of length at least one or two, commonly 3, 4, 5, 6, and possibly as much as 10 or even up to 20 residues (in the case of amino acids). In addition, such linkers need not be composed of amino acids but any oligomeric structures will do as well so long as they provide the correct spacing so as to optimize the desired level of immunogenic activity of the immunogens of the present invention. The immunogen may therefore take any form that is capable of eliciting a CTL response.

In addition, the immunogenic peptides of the present invention may be part of an immunogenic structure via attachments other than conventional peptide bonds. Thus, any manner of attaching the peptides of the invention to an immunogen of the invention, such as an immunogenic polypeptide as disclosed herein, could provide an immunogenic structure as claimed herein. Thus, immunogens, such as peptides of the invention, are structures that contain the peptides disclosed according to the present invention but such immunogenic peptides may not necessarily be attached thereto by the conventional means of using ordinary peptide bounds. The immunogens of the present invention simply contain such peptides as part of their makeup, but how such peptides are to be combined to form the final immunogen is left to the talent and imagination of the user and is in no way restricted or limited by the disclosure contained herein.

The peptides that are naturally processed and bound to a class I MHC molecule, and which are recognized by a tumor-specific CTL, need not be the optimal peptides for stimulating a CTL response. See, for example, (Parkhurst, M. R. et al., J.Immunol., 157:2539-2548, (1996); Rosenberg, S. A. et al., Nat.Med., 4:321-327, (1998)). Thus, there can be utility in modifying a peptide, such that it more readily induces a CTL response. Generally, peptides may be modified at two types of positions. The peptides may be modified at amino acid residues that are predicted to interact with the class I MHC molecule, in which case the goal is to create a peptide that has a higher affinity for the class I MHC molecule than does the parent peptide. The peptides can also be modified at amino acid residues that are predicted to interact with the T cell receptor on the CTL, in which case the goal is to create a peptide that has a higher affinity for the T cell receptor than does the parent peptide. Both of these types of modifications can result in a variant peptide that is related to a parent peptide, but which is better able to induce a CTL response than is the parent peptide.

The immunogenic peptides of the present invention, which may be used by themselves, or which are incorporated into oligopeptide or polypeptide immunogens of the invention, can be modified by the substitution of one or more residues at different, possibly selective, sites within the peptide chain. Using modern methods of peptide synthesis, amino acid substitutions can be made anywhere within the sequence of the peptide epitopes and such substitutions are by no means limited to the sequences disclosed herein. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

Conservative substitutions are herein defined as exchanges within one of the following five groups: Group 1—small aliphatic, nonpolar or slightly polar residues (Ala, Ser, Thr, Pro, Gly); Group 2—polar, negatively charged residues and their amides (Asp, Asn, Glu, Gin); Group 3—polar, positively charged residues (His, Arg, Lys); Group 4—large, aliphatic, nonpolar residues (Met, Leu, lie, Val, Cys); and Group 4—large, aromatic residues (Phe, Tyr, Trp).

Less conservative substitutions might involve the replacement of one amino acid by another that has similar characteristics but is somewhat different in size, such as replacement of an alanine by an isoleucine residue. Highly nonconservative replacements might involve substituting an acidic amino acid for one that is polar, or even for one that is basic in character. Such radical substitutions cannot, however, be dismissed as potentially ineffective since chemical effects are not totally predictable and radical substitutions might well give rise to serendipitous effects not otherwise predictable from simple chemical principles.

A comparison of the peptides of the present invention can also be made to the known peptide binding motifs for the respective class I MHC molecules. For example, the peptide binding motif for HLA-A3 is a leucine at position 2 of the peptide, and a tyrosine or lysine at the C-terminus of the peptide which is usually residue 9 (Engelhard, V. H., Ann.Rev.Immunol., 12:181-207, (1994)). To a lesser extent, phenylalanine is found at position 3 of the peptide. Thus, based on the known peptide binding motif for HLA-A3, a peptide of SEQ ID NO:4 can be constructed, in which the alanine at position 3 of the peptide with SEQ ID NO:1 is replaced with a phenylalanine.

Of course, such substitutions may involve structures other than the common L-amino acids. Thus, D-amino acids might be substituted for the L-amino acids commonly found in the antigenic peptides of the invention and yet still be encompassed by the disclosure herein. In addition, amino acids possessing non-standard R groups (i.e., R groups other than those found in the common 20 amino acids of natural proteins) may also be used for substitution purposes to produce immunogens and immunogenic polypeptides according to the present invention.

If substitutions at more than one position are found to result in a peptide with substantially equivalent or greater antigenic activity as defined below, then combinations of those substitutions will be tested to determine if the combined substitutions result in additive or syngeneic effects on the antigenicity of the peptide. At most, no more than 4 positions within the peptide would simultaneously be substituted.

Based on cytotoxicity assays, an epitope is considered substantially identical to the reference peptide if it has at least 10% of the antigenic activity of the reference peptide as defined by the ability of the substituted peptide to reconstitute the epitope recognized by a CTL in comparison to the reference peptide. Thus, when comparing the lytic activity in the linear portion of the effector:target curves with equimolar concentrations of the reference and substituted peptides, the observed percent specific killing of the target cells incubated with the substituted peptide should be equal to that of the reference peptide at an effector:target ratio that is no greater than 10-fold above the reference peptide effector:target ratio at which the comparison is being made.

Preferably, when the CTL specific for a peptide of SEQ ID NOs:1, 5-33 are tested against the substituted peptides, the peptide concentration at which the substituted peptides achieve half the maximal increase in lysis relative to background is no more than about 1 mM, preferably no more than about 1 μM, more preferably no more than about 1 nM, and still more preferably no more than about 100 pM, and most preferably no more than about 10 pM. It is also preferred that the substituted peptide be recognized by CTLs from more than one individual, at least two, and more preferably three individuals.

Thus, the epitopes of the present invention may be identical to naturally occurring tumor-associated or tumor-specific epitopes or may include epitopes that differ by no more than 4 residues from the reference peptide, as long as they have substantially identical antigenic activity.

It should be appreciated that an immunogen consisting only of a peptide of SEQ ID NO:1, or of a polypeptide that does not correspond to a protein of SEQ ID NO:2, but nonetheless comprises a peptide of SEQ ID NO:1, or of a plurality of peptides of SEQ ID NOs:1, 4-33, or of a polynucleotide coding for a plurality of peptides of SEQ ID NOs:1, 4-33 is necessarily limited to stimulating a CTL response that is specific for those peptides found in the immunogen. An advantage of using a polypeptide comprising SEQ ID NO:2 or a polynucleotide comprising SEQ ID NO:3 is that peptide epitopes that are naturally cleaved out of the c-ski oncogene-derived protein, and which are generated in addition to peptide epitopes of SEQ ID NOs:1, 5-33, can associate with an appropriate class I MHC molecules, which may or may not include HLA-A3. Thus, for example, immunogens comprising SEQ ID NO:2 and 3 find utility in the invention for stimulating CTL responses that are not limited to the recognition of cells that only express the combination of HLA- A3 and a peptide of SEQ ID NO:1.

The immunogens of the invention can also comprise a polypeptide that itself comprises one or more of the epitopic peptides of SEQ ID NOS:1, 4-33. Of course, such an immunogen could comprise c-ski itself (SEQ ID NO:2) or any of the peptides or polypeptides disclosed within the invention herein.

The immunogenic peptides and polypeptides of the invention can be prepared synthetically, by recombinant DNA technology, or they can be isolated from natural sources such as tumor cells expressing the c-ski oncogene protein product.

The peptides can be synthesized in solution or on a solid support in accordance with conventional techniques. Various automated peptide synthesizers are commercially available and can be used in accordance with known protocols. See, for example, (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York). Fragments of the polypeptide of the invention can also be synthesized as intermediates in the synthesis of a larger polypeptide.

Recombinant DNA technology may be employed wherein a nucleotide sequence which encodes an immunogenic peptide or polypeptide of interest is inserted into an expression vector, transformed or transfected into an appropriate host cell, and cultivated under conditions suitable for expression. These procedures are well known in the art to the skilled artisan, as described in (Coligan, J. E. et al, Current Protocols in Immunology, 1999, John Wiley & Sons, Inc., New York; Ausubel, F. M. et al, Current Protocols in Molecular Biology, 1999, John Wiley & Sons, Inc., New York; Molecular Cloning: A Laboratory Manual, 1989, Cold Spring Harbor Laboratory Press, Cold Spring Harbor). Thus, recombinantly produced peptides or polypeptides can be used as the immunogens of the invention.

The coding sequences for peptides of the length contemplated herein can be synthesized on commercially available automated DNA synthesizers using protocols that are well know in the art. See for example, (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York). The coding sequences can also be modified such that a peptide or polypeptide will be produced that incorporates a desired amino acid substitution. The coding sequence can be provided with appropriate linkers, be ligated into suitable expression vectors that are commonly available in the art, and the resulting DNA or RNA molecule can be transformed or transfected into suitable hosts to produce the desired fusion protein. A number of such vectors and suitable host systems are available, and their selection is left to the skilled artisan. For expression of the fusion proteins, the coding sequence will be provided with operably linked start and stop codons, promoter and terminator regions, and a replication system to provide an expression vector for expression in the desired host cell. For example, promoter sequences compatible with bacterial hosts are provided in plasmids containing convenient restriction sites for insertion of the desired coding sequence. The resulting expression vectors are transformed into suitable bacterial hosts. Of course, yeast, insect, and mammalian host cells may also be used, employing suitable vectors and control sequences.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pBluescript SK, PBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, PMSG, pSVL (Pharmacia). However, any other plasmid or vector may be used as long as they are replicable and viable in the host.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Ausubel, F. M. et al, Current Protocols in Molecular Biology, 1999, John Wiley & Sons, Inc., New York; Molecular Cloning: A Laboratory Manual, 1989, Cold Spring Harbor Laboratory Press, Cold Spring Harbor). Such cells can routinely be utilized for assaying CTL activity by having said genetically engineered, or recombinant, host cells express the immunogenic peptides of the present invention.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The immunogenic peptides of the present invention may be used to elicit CTLs ex vivo from either healthy individuals or from cancer patients with melanoma, colorectal carcinoma, lung carcinoma, ovarian carcinoma, or prostate carcinoma. Such responses are induced by incubating in tissue culture the individual's CTL precursor lymphocytes together with a source of antigen presenting cells and the appropriate immunogenic peptide. Examples of suitable antigen presenting cells include dendritic cells, macrophages, and activated B cells. Typically, the peptide at concentrations between 10 and 40 μg/ml, would be pre-incubated with the antigen presenting cells for periods ranging from 1 to 18 hrs. β₂-microglobulin (4 μg/ml) can be added during this time period to enhance binding. The antigen presenting cells may also be held at room temperature during the incubation period (Ljunggren, H.-G. et al., Nature, 346:476480, (1990)) or pretreated with acid (Zeh, H. J., III et al., Hum.Immunol., 39:79-86, (1994)) to promote the generation of denatured class I MHC molecules which can then bind the peptide. The precursor CTLs (responders) are then added to the antigen presenting cells to which the immunogenic peptide has bound (stimulators) at responder to stimulator ratios of between 5:1 and 50:1, and most typically between 10:1 and 20:1. The co-cultivation of the cells is carried out at 37° C. in RPMI 1640, 10% fetal bovine serum, 2 mM L-glutamine, and IL-2 (5-20 Units/ml). Other cytokines, such as IL-1, IL-7, and IL-12 may also be added to the culture. Fresh IL-2-containing media is added to the cultures every 2-4 days, typically by removing one-half the old media and replenishing it with an equal volume of fresh media. After 7-10 days, and every 7-10 days thereafter, the CTL are restimulated with antigen presenting cells to which immunogenic peptide has been bound as described above. Fresh IL-2-containing media is added to the cells throughout their culture as described above. Three to four rounds of stimulation, and sometimes as many five to eight rounds of stimulation, are required to generate a CTL response that can then be measured in vitro. The above described protocol is illustrative only and should not be considered limiting. Many in vitro CTL stimulation protocols have been described and the choice of which one to use is well within the knowledge of the skilled artisan. The peptide-specific CTL can be further expanded to large numbers by treatment with anti-CD3 antibody. For example, see (Riddell, S. R. and Greenberg, P. D., J.Immunol.Methods, 128:189-201, (1990); Walter, E. A. et al., N.Engl.J.Med., 333:1038-1044, (1995)).

Antigen presenting cells that are to be used to stimulate a CTL response are typically incubated with peptide of an optimal length, most commonly a nonapeptide, that allows for direct binding of the peptide to the class I MHC molecule without additional processing. Larger oligopeptides and polypeptides are generally ineffective in binding to class I MHC molecules as they are not efficiently processed into an appropriately sized peptide in the extracellular milieu. There a variety of approaches that are known in the art, however, that allow oligopeptides and polypeptides to be exogenously acquired by a cell, which then allows for their subsequent processing and presentation by a class I MHC molecule. Representative, but non-limiting examples of such approaches include electroporation of the molecules into the cell (Harding, C. H. III, Eur.J.Immunol., 22:1865-1869, (1992)), encapsulation of the molecules in liposomes which are fused to the cells of interest (Reddy, R. et al., J.Immunol.Methods, 141:157-163, (1991)), or osmotic shock in which the molecules are taken up via pinocytosis (Moore, M. W. et al., Cell, 54:777-785, (1988)). Thus, oligopeptides and polypeptides that comprise one or more of the peptides of the invention can be provided to antigen presenting cells in such a fashion that they are delivered to the cytoplasm of the cell, and are subsequently processed to allow presentation of the peptides.

Antigen presenting cells suitable for stimulating an in vitro CTL response that is specific for one or more of the peptides of the invention can also be prepared by introducing polynucleotide vectors encoding the sequences into the cells. These polynucleotides can be designed such that they express only a single peptide of the invention, multiple peptides of the invention, or even a plurality of peptides of the invention. There are a variety of approaches that are known in the art, that allow polynucleotides to be introduced and expressed in a cell, thus providing one or more peptides of the invention to the class I MHC molecule binding pathway. Representative, but non-limiting examples of such approaches include the introduction of plasmid DNA through particle-mediated gene transfer or electroporation (Tuting, T. et al., J.Immunol., 160:1139-1147, (1998)), or the transduction of cells with an adenovirus expressing the polynucleotide of interest (Perez-Diez, A. et al., Cancer Res., 58:5305-5309, (1998)). Thus, oligonucleotides that code for one or more of the peptides of the invention can be provided to antigen presenting cells in such a fashion that the peptides associate with class I MHC molecules and are presented on the surface of the antigen presenting cell, and consequently are available to stimulate a CTL response.

By preparing the stimulator cells used to generate an in vitro CTL response in different fashions, it is possible to control the peptide specificity of CTL response. For example, the CTLs generated with a particular peptide will necessarily be specific for that peptide. Likewise, CTLs that are generated with a polypeptide or polynucleotide expressing or coding for particular peptides will be limited to specificities that recognize those peptides. More broadly, stimulator cells, and more specifically dendritic cells, can be incubated in the presence of the whole c-ski oncogene protein (SEQ ID NO:2). As a further alternative, stimulator cells, and more specifically dendritic cells, can be transduced or transfected with RNA or DNA comprising the polynucleotide sequence of SEQ ID NO:3. Under these alternative conditions, peptide epitopes that are naturally cleaved out of the c-ski oncogene-derived protein, and which are generated in addition to peptide epitopes of SEQ ID NOs:1 or 5-33, can associate with an appropriate class I MHC molecule, which may or may not include HLA-A1, -A2, or -A3. Thus, for example, immunogens of SEQ ID NO:2 and 3 find use in the invention for stimulating CTL responses that are not limited to the recognition of cells that only express the combination of HLA-A3 and a peptide of SEQ ID NO:1. The selection of antigen presenting cells and the type of antigen with which to stimulate the CTL with, is left to the ordinary skilled artisan.

In specific embodiments, the methods of the present invention include a method for inducing a CTL response in vitro that is specific for a tumor cell expressing HLA-A1, -A2, or -A3, and the c-ski oncogene (SEQ ID NO:3), whereby the method comprises contacting a CTL precursor lymphocyte with an antigen presenting cell that has bound an immunogenic peptide selected from a group comprising the peptides disclosed according to the invention.

In specific embodiments, the methods of the present invention include a method for inducing a CTL response in vitro that is specific for a tumor cell expressing HLA-A1, -A2, or -A3, and the c-ski oncogene (SEQ ID NO:3), whereby the method comprises contacting a CTL precursor lymphocyte with an antigen presenting cell that has exogenously acquired an immunogenic oligopeptide or polypeptide that comprises one or more of the peptides disclosed according to the invention.

An additional embodiment relates to a method of inducing a CTL response in vitro that is specific for a tumor cell expressing the c-ski oncogene (SEQ ID NO:3), whereby the method comprises contacting a CTL precursor lymphocyte with an antigen presenting cell that is expressing a polynucleotide of SEQ ID NO: 3.

A yet additional embodiment of the present invention is directed to a method of inducing a CTL response in vitro that is specific for a tumor cell expressing HLA-A1, -A2, or -A3, and the c-ski oncogene (SEQ ID NO:3), whereby the method comprises contacting a CTL precursor lymphocyte with an antigen presenting cell that is expressing a polynucleotide coding for at least one CTL epitope and wherein the epitope is an epitopic peptide of the invention, the polynucleotide is operably linked to a promoter and wherein the polynucleotide sequence does not include the entire c-ski gene of SEQ ID NO:2.

A variety of techniques exist for assaying the activity of CTL. These techniques include the labeling of target cells with radionuclides such as Na₂⁵¹CrO₄ or ³H-thymidine, and measuring the release or retention of the radionuclides from the target cells as an index of cell death. Such assays are well-known in the art and their selection is left to the skilled artisan. Alternatively, CTL are known to release a variety of cytokines when they are stimulated by an appropriate target cell, such as a tumor cell expressing the relevant class I MHC molecule and the corresponding peptide. Non-limiting examples of such cytokines include IFN-γ, TNFα, and GM-CSF. Assays for these cytokines are well known in the art, and their selection is left to the skilled artisan. Methodology for measuring both target cell death and cytokine release as a measure of CTL reactivity are given in (Coligan, J. E. et al, Current Protocols in Immunology, 1999, John Wiley & Sons, Inc., New York).

After expansion of the antigen-specific CTL, the cells can then be adoptively transferred back into the patient, where they will destroy their specific target cell. The utility of such adoptive transfer is demonstrated in (North, R. J. et al., Infect.Immun., 67:2010-2012, (1999); Riddell, S. R. et al., Science, 257:238-241, (1992)). In determining the amount of cells to reinfuse, the skilled physician will be guided by the total number of cells available, the activity of the CTL as measured in vitro, and the condition of the patient. Preferably, however, about 1×10⁶ to about 1×10¹², more preferably about 1×10⁸ to about 1×1011, and even more preferably, about 1×10⁹ to about 1×10¹⁰ peptide-specific CTL are infused. Methodology for reinfusing the T cells into a patient are well known and exemplified in U.S. Pat. No. 4,844,893 to Honski, et al., and U.S. Pat. No. 4,690,915 to Rosenberg.

The peptide-specific CTL can be purified from the stimulator cells prior to infusion into the patient. For example, monoclonal antibodies directed towards the cell surface protein CD8, present on CTL, can be used in conjunction with a variety of isolation techniques such as antibody panning, flow cytometric sorting, and magnetic bead separation to purify the peptide-specific CTL away from any remaining non-peptide specific lymphocytes or from the stimulator cells. These methods are well-known in the art, and are their selection is left to the skilled artisan. It should be appreciated that generation of peptide-specific CTL in this manner, obviates the need for stimulating the CTL in the presence of tumor. Thus, there is no chance of inadvertently reintroducing tumor cells into the patient.

Thus, one embodiment of the present invention relates to a method of treating a subject with cancer characterized by tumor cells expressing complexes of HLA-A1, -A2, and -A3 and the c-ski oncogene (SEQ ID NO:3), whereby CTLs produced in vitro according to the present invention are administered in an amount sufficient to destroy the tumor cells through direct lysis or to effect the destruction of the tumor cells indirectly through the elaboration of cytokines.

Another embodiment of the present invention is directed to a method of treating a subject with cancer characterized by tumor cells expressing any class I MHC molecule and a gene of SEQ ID NO:3, whereby the CTLs are produced in vitro and are specific for the c-ski oncogene and are administered in an amount sufficient to destroy the tumor cells through direct lysis or to effect the destruction of the tumor cells indirectly through the elaboration of cytokines.

In the foregoing embodiments the cancer to be treated includes a melanoma, a colorectal carcinoma, an ovarian carcinoma, a lung carcinoma, and prostate carcinoma.

The ex vivo generated CTL can be used to identify and isolate the T cell receptor molecules specific for the peptide. The genes encoding the alpha and beta chains of the T cell receptor can be cloned into an expression vector system and transferred and expressed in naïve T cells from peripheral blood, T cells from lymph nodes, or T lymphocyte progenitor cells from bone marrow. These T cells, which would then be expressing a peptide-specific T cell receptor, would then have anti-tumor reactivity and could be used in adoptive therapy of cancer, and more specifically melanoma, colorectal carcinoma, ovarian carcinoma, lung carcinoma, and prostate carcinoma.

In addition to their use for therapeutic or prophylactic purposes, the immunogenic peptides of the present invention are useful as screening and diagnostic agents. Thus, the immunogenic peptides of the present invention, together with modern techniques of gene screening, make it possible to screen patients for the presence of genes encoding of such peptides on cells obtained by biopsy of tumors detected in such patients. The results of such screening may help determine the efficacy of proceeding with the regimen of treatment disclosed herein using the immunogens of the present invention.

Alternatively, the immunogenic peptides disclosed herein, as well as functionally similar homologs thereof, may be used to screen a sample for the presence of CTLs that specifically recognize the corresponding epitopes. The lymphocytes to be screened in this assay will normally be obtained from the peripheral blood, but lymphocytes can be obtained from other sources, including lymph nodes, spleen, tumors, and pleural fluid. The peptides of the present invention may then be used as a diagnostic tool to evaluate the efficacy of the immunotherapeutic treatments disclosed herein. Thus, the in vitro generation of CTL as described above would be used to determine if patients are likely to respond to the peptide in vivo. Similarly, the in vitro generation of CTL could be done with samples of lymphocytes obtained from the patient before and after treatment with the peptides. Successful generation of CTL in vivo should then be recognized by a correspondingly easier ability to generate peptide-specific CTL in vitro from lymphocytes obtained following treatment in comparison to those obtained before treatment.

The peptides of the invention can also be used to prepare class I MHC tetramers which can be used in conjunction with flow cytometry to quantitate the frequency of peptide-specific CTL that are present in a sample of lymphocytes from an individual. Specifically, for example, class I MHC molecules comprising HLA-A3 and peptides of SEQ ID NO:1 would be combined to form tetramers as exemplified in U.S. Pat. No. 5,635,363. Said tetramers would find use in monitoring the frequency of CTL specific for the combination of HLA-A3 and a peptide of SEQ ID NO:1 in the peripheral blood, lymph nodes, or tumor mass of an individual undergoing immunotherapy with the peptides, proteins, or polynucleotides of the invention, and it would be expected that successful immunization would lead to an increase in the frequency of the peptide-specific CTL. Said tetramers could also be developed for peptides of SEQ ID NOs:5-14 in combination with HLA-A1, SEQ ID NOs:15-24 in combination with HLA-A2, or SEQ ID NOs:25-33 in combination with HLA-A3.

As indicated above, a vaccine in accordance with the present invention may include one or more of the hereinabove described polypeptides or active fragments thereof, or a composition, or pool, of immunogenic peptides disclosed herein. When employing more than one polypeptide or active fragment, such as two or more polypeptides and/or active fragments may be used as a physical mixture or as a fusion of two or more polypeptides or active fragments. The fusion fragment or fusion polypeptide may be produced, for example, by recombinant techniques or by the use of appropriate linkers for fusing previously prepared polypeptides or active fragments.

The immunogenic molecules of the invention, including vaccine compositions, may be utilized according to the present invention for purposes of preventing, suppressing or treating diseases causing the expression of the immunogenic peptides disclosed herein, such as where the antigen is being expressed by tumor cells. As used in accordance with the present invention, the term “prevention” relates to a process of prophylaxis in which an animal, especially a mammal, and most especially a human, is exposed to an immunogen of the present invention prior to the induction or onset of the disease process. This could be done where an individual has a genetic pedigree indicating a predisposition toward occurrence of the disease condition to be prevented. For example, this might be true of an individual whose ancestors show a predisposition toward certain types of cancer. Alternatively, the immunogen could be administered to the general population as is frequently done for infectious diseases. Alternatively, the term “suppression” is often used to describe a condition wherein the disease process has already begun but obvious symptoms of said condition have yet to be realized. Thus, the cells of an individual may have become cancerous but no outside signs of the disease have yet been clinically recognized. In either case, the term prophylaxis can be applied to encompass both prevention and suppression. Conversely, the term “treatment” is often utilized to mean the clinical application of agents to combat an already existing conditions whose clinical presentation has already been realized in a patient. This would occur where an individual has already been diagnosed as having a tumor.

It is understood that the suitable dosage of an immunogen of the present invention will depend upon the age, sex, health, and weight of the recipient, the kind of concurrent treatment, if any, the frequency of treatment, and the nature of the effect desired. However, the most preferred dosage can be tailored to the individual subject, as determined by the researcher or clinician. The total dose required for any given treatment will commonly be determined with respect to a standard reference dose based on the experience of the researcher or clinician, such dose being administered either in a single treatment or in a series of doses, the success of which will depend on the production of a desired immunological result (i.e., successful production of a CTL-mediated response to the antigen, which response gives rise to the prevention and/or treatment desired). Thus, the overall administration schedule must be considered in determining the success of a course of treatment and not whether a single dose, given in isolation, would or would not produce the desired immunologically therapeutic result or effect. The therapeutically effective amount of a composition containing one or more of the immunogens of this invention, is an amount sufficient to induce an effective CTL response to the antigen and to cure or arrest disease progression. Thus, this dose will depend, among other things, on the identity of the immunogens used, the nature of the disease condition, the severity of the disease condition, the extent of any need to prevent such a condition where it has not already been detected, the manner of administration dictated by the situation requiring such administration, the weight and state of health of the individual receiving such administration, and the sound judgment of the clinician or researcher. Thus, for purposes of prophylactic or therapeutic administration, effective amounts would generally lie within the range of from 1.0 pg to about 5,000 μg of peptide for a 70 kg patient, followed by boosting dosages of from about 1.0 μg to about 1,000 μg of peptide pursuant to a boosting regimen over days, weeks or even months, depending on the recipient's response and as necessitated by subsequent monitoring of CTL-mediated activity within the bloodstream. Of course, such dosages are to be considered only a general guide and, in a given situation, may greatly exceed such suggested dosage regimens where the clinician believes that the recipient's condition warrants more a aggressive administration schedule. Needless to say, the efficacy of administering additional doses, and of increasing or decreasing the interval, may be re-evaluated on a continuing basis, in view of the recipient's immunocompetence (for example, the level of CTL activity with respect to tumor-associated or tumor-specific antigens).

For such purposes, the immunogenic compositions according to the present invention may be used against a disease condition such as cancer by administration to an individual by a variety of routes. The composition may be administered parenterally or orally, and, if parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time.

Generally, vaccines are prepared as injectables, in the form of aqueous solutions or suspensions. Vaccines in an oil base are also well known such as for inhaling. Solid forms which are dissolved or suspended prior to use may also be formulated. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. These compositions may be sterilized by conventional, well known sterilization techniques including sterile filtration. The resulting solutions may be packaged for use as is, or the acqueous solutions may be lyophilized, the lyophilized preparation being combined with sterile water before administration. Vaccine compositions may further incorporate additional substances to stabilize pH, or to function as adjuvants, wetting agents, or emulsifying agents, which can serve to improve the effectiveness of the vaccine.

The concentration of the CTL stimulatory peptides of the invention in pharmaceutical formulations are subject to wide variation, including anywhere from less than 0.01% by weight to as much as 50% or more. Factors such as volume and viscosity of the resulting composition must also be considered. The solvents, or diluents, used for such compositions include water, possibly PBS (phosphate buffered saline), or saline itself, or other possible carriers or excipients.

The immunogens of the present invention may also be contained in artificially created structures such as liposomes, ISCOMS, slow-releasing particles, and other vehicles which increase the immunogenicity and/or half-life of the peptides or polypeptides in serum. Liposomes include emulsions, foams, micelies, insoluble monolayers, liquid crystals, phospholipid dispersions, lamellar layers and the like. Liposomes for use in the invention are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by (Coligan, J. E. et al, Current Protocols in Protein Science, 1999, John Wiley & Sons, Inc., New York)and see also U.S. Pat. Nos. 4,235,871, 4,501,728, 4,837,028, and 5,019,369.

Liposomes containing the peptides or polypeptides of the invention can be directed to the site of lymphoid cells where the liposomes then deliver the selected immunogens directly to antigen presenting cells. Targeting can be achieved by incorporating additional molecules such as proteins or polysaccharides into the the outer membranes of said structures, thus resulting in the delivery of the structures to particular areas of the body, or to particular cells within a given organ or tissue. Such targeting molecules may a molecule that binds to receptor on antigen presenting cells. For example an antibody that binds to CD80 could be used to direct liposomes to dendritic cells.

The immunogens of the present invention may also be administered as solid compositions. Conventional nontoxic solid carriers including pharmaceutical grades of mannitol, lactose, starch, magnesium, cellulose, glucose, sucrose, sodium saccharin, and the like. Such solid compositions will often be administered orally, whereby a pharmaceutically acceptable nontoxic composition is formed by incorporating the peptides and polypeptides of the invention with any of the carriers listed above. Generally, such compositions will contain 10-95% active ingredient, and more preferably 25-75% active ingredient.

Aerosol administration is also an alternative, requiring only that the immunogens be properly dispersed within the aerosol propellant. Typical percentages of the peptides or polypeptides of the invention are 0.01% -20% by weight, preferably 1% -10%. The use of a surfactant to properly disperse the immunogen may be required. Representative surfactants include the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1% -20% by weight of the composition, preferably 0.25-5%. Typical propellants for such administration may include esters and similar chemicals but are by no means limited to these. A carrier, such as lecithin for intranasal delivery, may also be included.

The peptides and polypeptides of the invention may also be delivered with an adjuvant. Adjuvants include, but are not limited to complete or incomplete Freund's adjuvant, Montanide ISA-51, aluminum phosphate, aluminum hydroxide, alum, and saponin. Adjuvant effects can also be obtained by injecting a variety of cytokines along with the immunogens of the invention. These cytokines include, but are not limited to IL-1, IL-2, IL-7, IL-12, and GM-CSF.

The peptides and polypeptides of the invention can also be added to professional antigen presenting cells such as dendritic cells that have been prepared ex vivo. For example, the dendritic cells could be prepared from CD34 positive stem cells from the bone marrow, or they could be prepared from CD14 positive monocytes obtained from the peripheral blood. The dendritic cells are generated ex vivo using cytokines such as GM-CSF, IL-3, IL-4, TNF, and SCF. The cultured DC are then pulsed with peptides at various concentrations using standard methods that are well known in the art. The peptide-pulsed dendritic cells can then be administered either intraveneously, subcutaneously, or intradermally , and the immunization may also include cytokines such as IL-2 or IL-12.

The present invention is also directed to a vaccine in which a peptide or polypeptide or active fragment of the present invention is delivered or administered in the form of a polynucleotide encoding the peptide or polypeptide or active fragment, whereby the peptide or polypeptide or active fragment is produced in vivo. The polynucleotide may be included in a suitable expression vector and combined with a pharmaceutically acceptable carrier. For example, the peptides or polypeptides could be expressed in plasmid DNA and nonreplicative viral vectors such as vaccinia, fowlpox, Venezuelan equine encephalitis virus, adenovirus, or other RNA or DNA viruses. These examples are meant to be illustrative only and should not be viewed as self-limiting A wide variety of other vectors are available and are apparent to those skilled in the art from the description given herein. In this approach, a portion of the nucleotide sequence of the viral vector is engineered to express the peptides or polypeptides of the invention. Vaccinia vectors and methods useful in immunization protocols are described in U.S. Pat. No. 4,722,848, the methods of which are incorporated here by reference.

Regardless of the nature of the composition given, additional therapeutic agents may also accompany the immunogens of the present invention. Thus, for purposes of treating tumors, compositions containing the immunogens disclosed herein may, in addition, contain other antitumor pharmaceuticals. The use of such compositions with multiple active ingredients is left to the discretion of the clinician.

In addition, the immunogenic polypeptides and peptides of the present invention can be used to stimulate the production of antibodies for use in passive immunotherapy, for use as diagnostic reagents, and for use as reagents in other processes such as affinity chromatography.

A specific embodiment of the present invention relates to a method for inducing a CTL response in a subject, wherein the immunogen is in the form of one or more peptides. The method comprises administering to subjects that express HLA-A1, -A2, or -A3, at least one CTL epitope, wherein said epitope or epitopes are selected from a group comprising the peptides disclosed according to the invention, in an amount sufficient to induce a CTL response to tumor cells expressing at least one of HLA-A1, -A2, or -A3, and the c-ski oncogene (SEQ ID NO:3).

A further embodiment of the present invention relates to a method for inducing a CTL response in a subject, wherein the immunogen is in the form of one or more oligopeptides or polypeptides. The method comprises administering to subjects that express HLA-A1, -A2, or -A3, at least one CTL epitope, wherein said epitope or epitopes are selected from a group comprising the peptides disclosed according to the invention, and are contained within oligopeptides or polypeptides that do not comprise the entire c-ski polypeptide (SEQ ID NO:2), in an amount sufficient to induce a CTL response to tumor cells expressing at least one of HLA-A1, -A2, or -A3, and the c-ski oncogene (SEQ ID NO:3).

A still further embodiment of the present invention relates to a method for inducing a CTL response in a subject, wherein the immunogen is in the form of a polynucleotide. The method comprises administering to subjects that express HLA-A1, -A2, or -A3, at least one CTL epitope, wherein said epitope or epitopes are selected from a group comprising the peptides disclosed according to the invention, and are coded within a polynuclotide sequence that does not comprise the entire c-ski oncogene (SEQ ID NO:3), in an amount sufficient to induce a CTL response to tumor cells expressing at least one of HLA-A1, -A2, or -A3, and the c-ski oncogene (SEQ ID NO:3).

While the below examples are provided to illustrate the invention, it is to be understood that these methods and examples in no way limit the invention to the embodiments described herein and that other embodiments and uses will no doubt suggest themselves to those skilled in the art. All publications, patents, and patent applications cited herein are hereby incorporated by reference, as are the references cited therein. It is also to be understood that throughout this disclosure where the singular is used, the plural may be inferred and vice versa and use of either is not to be considered limiting.

### EXAMPLE 1

### Cell Lines

VMM18, a malignant melanoma, was previously established in culture (Skipper, J. C. et al., J.Immunol., 157:5027-5033, (1996)), and was maintained in RPMI1640 supplemented with 10% fetal bovine serum (FBS) and 2 mM L-glutamine. Hmy2.C1R, a class I MHC-negative B-lymphoblastoid cell line, was previously transfected with the gene encoding HLA-A3. The resulting line, C1R-A3 was maintained in RPMI1640 supplemented with 10% FBS, 2 mM L-glutamine, and 300 μg/ml G418.

### EXAMPLE 2

### Immunoaffinity Purification

VMM18 cells were grown in 10-chamber Nunc cell factories (Fisher, Pittsburgh, Pa.). The cells were harvested by treatment with 0.45% trypsin and 0.32 mM EDTA, washed two times in phosphate-buffered saline solution (pH 7.4), and stored as cell pellets at -80° C. Aliquots of 6-8×10¹⁰ cells were solubilized at 5-10×10⁶ cells/ml in 20 mM Tris, pH 8.0, 150 mM NaCl, 1% CHAPS, 18.5 μg/ml iodoacetamide, 5 μg/ml aprotonin, 10 μg/ml leupeptin, 10 μg/ml pepstatin A, 5 mM EDTA, 0.2% sodium azide, and 17.4 μg/ml phenylmethylsulfonyl fluoride for 1 h. This and all subsequent steps were performed with ice-cold solutions and at 4° C. The lysates were then centrifuged at 100,000×g, the pellets discarded, and the supernatants passed through a 0.22 μm filter. The supernatants were then passed over a series of columns with the first containing Sepharose, and the second containing the HLA-A3-specific monoclonal antibody, GAP-A3, bound to a protein A-Sepharose matrix. The second column was then sequentially washed with 20 column volumes of 20 mM Tris, pH 8.0, 150 mM NaCl, 20 column volumes of 20 mM Tris, pH 8.0, 1.0 M NaCl, and 20 column volumes of 20 mM Tris, pH 8.0. The peptides were eluted from the column with 5 column volumes of 10% acetic acid. The isolated HLA-A3 molecules were then boiled for 5 min to further dissociate any bound peptide from the heavy chains. The peptides were then separated from the co-purifying class I heavy chain and β₂-microglobulin by centrifugation on a Ultrafree-CL membrane with a nominal molecular weight cut-off of 5,000 Daltons (Millipore, Beford, Mass.).

### EXAMPLE 3

### Peptide Fractionation

The peptide extracts were fractionated by RP-HPLC (Reversed Phase—High Performance Liquid Chromatography) using an Applied Biosystems (ABI) model 140B system. The extracts were concentrated by vacuum centrifugation from about 20 ml down to 250 μl and injected into either a Brownlee (Norwalk, Conn.) C₁₈ Aquapore column (2.1 mm×3 cm; 300 Å; 7 μm) or a Higgins (Mountain View, Calif.) C18 Haisil column (2.1 mm×4 cm; 300 Å; 5 μm). The peptides were eluted by first using a gradient of acetonitrile/0.085% TFA (trifluoroacetic acid) in 0.1% TFA/water, with the concentration of acetonitrile increasing from 0-9% (0-5 minutes), 9-36% (5-55 minutes), and 36-60% (55-62 minutes). A second dimension fractionation of combined fractions 17 and 18 from the first dimension (TFA) fraction was accomplished using the same gradient but with the substitution of HFBA (heptafluorobutyric acid) for TFA. The flow rate was 200 μl/min, and fractions were collected at 1 min (Brownlee column) or 40 second (Higgins column) intervals. A third dimension of RP-HPLC was achieved using an Eldex (Napa, Calif.) MicroPro Pump, a homemade C₁₈ microcapillary column, and an ABI model 785A UV absorbance detector. The column was prepared by packing a 27 cm bed of 10 μm C₁₈ particles in a section of 285 μm o.d./75 μm i.d. fused silica (Polymicro Technologies, Phoenix, Ariz.). Peptides in combined fractions 26 and 27 of the second dimension fraction were loaded onto this column and eluted with a gradient of acetonitrile/0.67% triethylamine acetate/water in 0.1% triethylamine acetate/water, with the concentration of acetonitrile increasing from 0-60% in 40 minutes. The flow rate was about 300 nl/min, and fractions were collected into 25 μl of water every 30 s. In all RP-HPLC experiments, peptides were detected by monitoring UV absorbance at 214 nm.

### EXAMPLE 4

### Mass Spectrometric Analysis

The second dimension HPLC fraction was analyzed using an affluent splitter on the microcapillary HPLC column. In this experiment, the column (360 μm o.d.×100 μm i.d. with a 25 cm C₁₈ bed) was butt connected with a zero dead volume tee (Valco, Houston, Tex.) to two pieces of fused silica of different lengths (25 μm and 40 μm i.d.). Peptides were eluted with a 34 min gradient of 0-60% acetonitrile. The 25 μm capillary deposited one-fifth of the HPLC effluent into the wells of a microtitre plate for use in CTL epitope reconstitution assays, whereas the remaining four-fifths of the effluent was directed into the mass spectrometer. Ions were formed by electrospray ionization, and mass spectra were recorded by scanning between mass to charge ratios (m/z) 300 and 1400 every 1.5 seconds. Peptide sequences were determined by CAD (collision-activated dissociation) tandem mass spectrometry as described in the literature (Hunt, D. F. et al., Proc.Natl.Acad.Sci.U.S.A, 83:6233-6237, (1986)).

### EXAMPLE 5

### Peptide Synthesis

Peptides were synthesized using a Gilson (Madison, Wis.) AMS 422 multiple peptide synthesizer. Ten pMol quantities were synthesized using conventional FMOC amino acids, resins, and chemical techniques. Peptides were purified by RP-HPLC using a 4.6 mm×100 mm POROS (Perseptive Biosystems, Cambridge, Mass.) column and a 10 min, 0-60% acetonitrile in 0.1% TFA gradient.

### EXAMPLE 6

### Identification of a c-ski-oncogene-derived Peptide that Associates with HLA-A3.

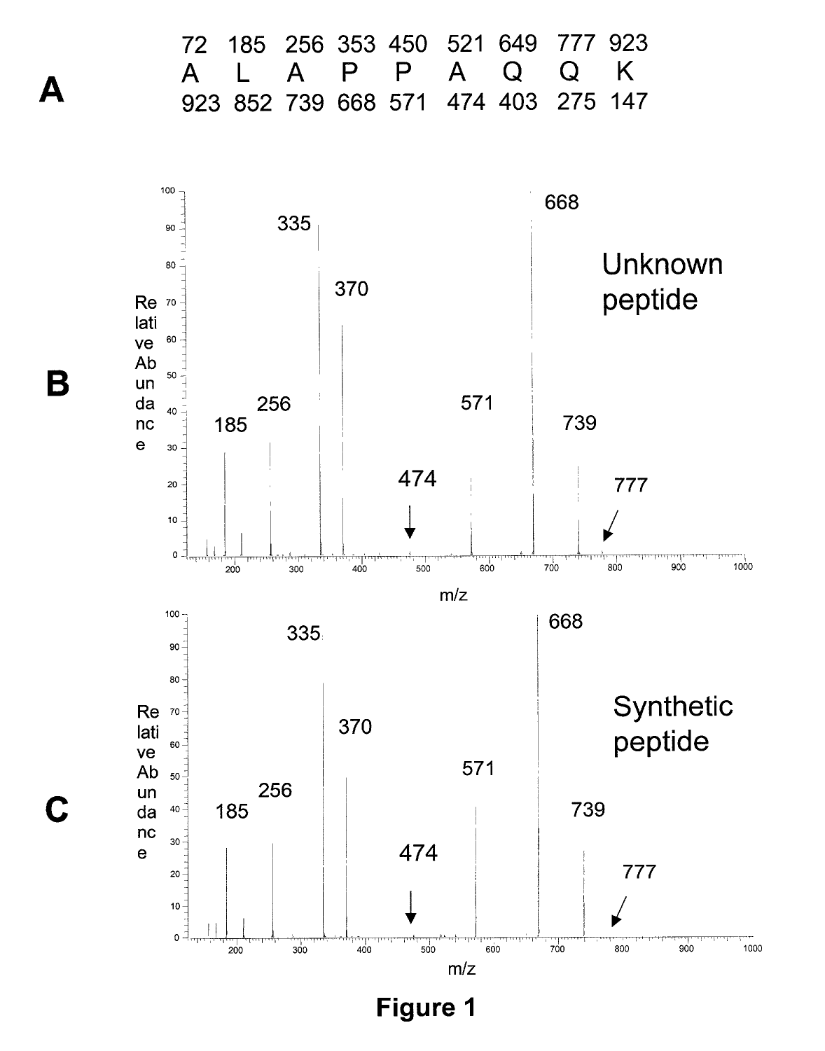

To identify the antigens present on the surface of the VMM18 melanoma tumor cell line, HLA-A3 molecules were purified by immunoaffinity chromatography from 8×10¹⁰ VMM18 tumor cells. After acid elution of the affinity column and dissociation of the class I/peptide complexes by boiling, the peptides were separated from the HLA-A3 heavy chains and β2-microglobulin by membrane filtration, concentrated, and then fractionated by RP-HPLC using TFA as the organic modifier. Fractions 17 and 18 of the first dimension fractionation were combined, and subjected to a further round of fractionation using HFBA as the organic modifier. Fractions 26 and 27 of the second dimension fractionation were then combined, and subjected to a third round of fractionation using TEAA as the organic modifier. An ion with a mass to charge ration (m/z) of 462.62²⁺ in fraction 24 was selected for sequence analysis by collision activated dissociation (CAD). Analysis of the fragmented masses obtained from the CAD allowed the determination of the peptide sequence as ALAPPAQQK (SEQ ID NO:1) (FIG. 1A). CAD of a synthetic peptide corresponding to SEQ ID NO:1 unequivocally identified the unknown as having the sequence of SEQ ID NO:1 (FIG. 1B). A Genbank search revealed that a peptide of SEQ ID NO:1 corresponds exactly to amino acid residues 420 to 428 of the c-ski oncogene (SEQ ID NO:2). The c-ski oncogene is in turn coded for by a cDNA corresponding to SEQ ID NO:3.

### EXAMPLE 7

### Expression of the c-ski Oncogene cDNA in Malignant and Healthy Tissue by RT-PCR Analysis

Total RNA was purified from PBS washed snap-frozen cell pellets (approximately 1×10⁶ cells per sample) of various tumor cell lines using RNAzol™B (Tel-Test Inc, Friendswood, Tex.). Purified total RNA was ethanol precipitated and quantified by absorbence at 260 nm. First strand cDNA was synthesized by reverse transcribing 2 μg of total RNA with oligo(dT) using the SuperScript™ system (Gibco BRL, Gaithersburg, Md.). Buffer mix G from the FailSafe™ PCR Premix system (Epicentre Technologies, Madison, Wis.) was used for polymerase chain reaction (PCR) amplification of 1 μl of newly made first strand cDNA. In some instances, a human multiple tissue cDNA panel (MTC™, Clontech, Palo Alta, Calif.) was also used as a template source for c-ski specific PCR amplification.

Oligonucleotide primers for RT-PCR amplification of a portion of the human c-ski oncogene (accession# X15218) were synthesized by IDT (Integrated DNA Technologies Inc.,Coralville, Iowa). Primers Nski1 (coding strand, position 2770 to 2789; 5′-TTC AGC GAC MG GAC CCA GG) and Nski2 (non-coding strand, position 3161 to 3142; 5′-GTT CCA ATG CTG CAG CCT CA) were used to amplify a 392 bp PCR product from first strand cDNA using the following procedure: After heating PCR components to 94° C., 1 μl of cDNA was added to each tube. Subsequent cycling conditions were 94° C. for 30 s, 55° C. for 1 min, and 72° C. for 1 min for 30 or 35 cycles with a final extension of 72° C. for 10 min. PCR products were analyzed on ethidium bromide stained agarose gels.

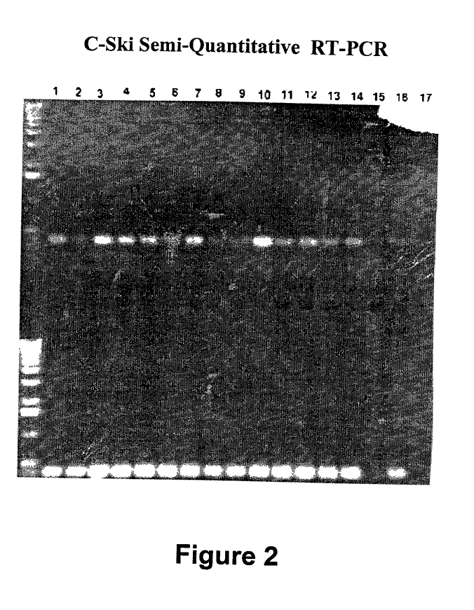

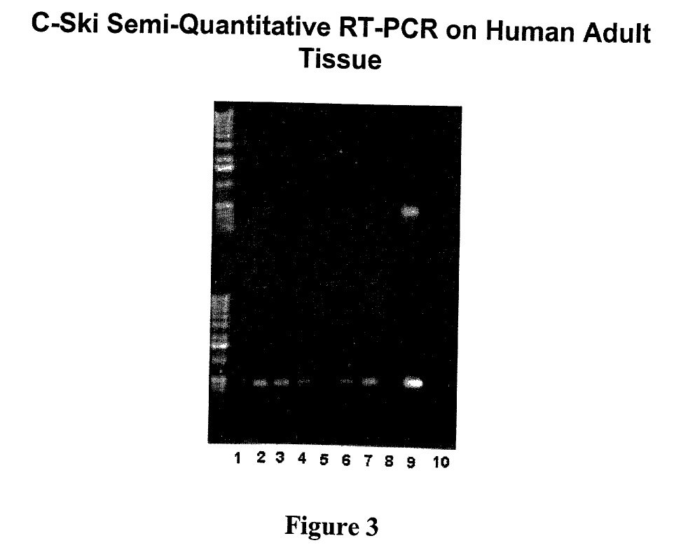

RT-PCR with c-ski-specific primers was performed on 15 different melanoma tumor lines and 1 B-lymphoblastoid cell line (FIG. 2). This analysis indicated that the c-ski oncogene is expressed at varying levels in all of the melanoma lines tested, and that the oncogene is not expressed in a B cell line. Further analysis was performed on cDNA derived from 8 different normal tissues (FIG. 3). This analysis indicated that the c-ski-oncogene was not expressed or was expressed at levels significantly lower than that found in a melanoma line. This data is consistent with previously published results in which it was shown that c-ski was over-expressed in melanoma cell lines relative to normal melanocytes (Fumagalli, S et al., Melanoma Res., 3:23-27, (1993)).

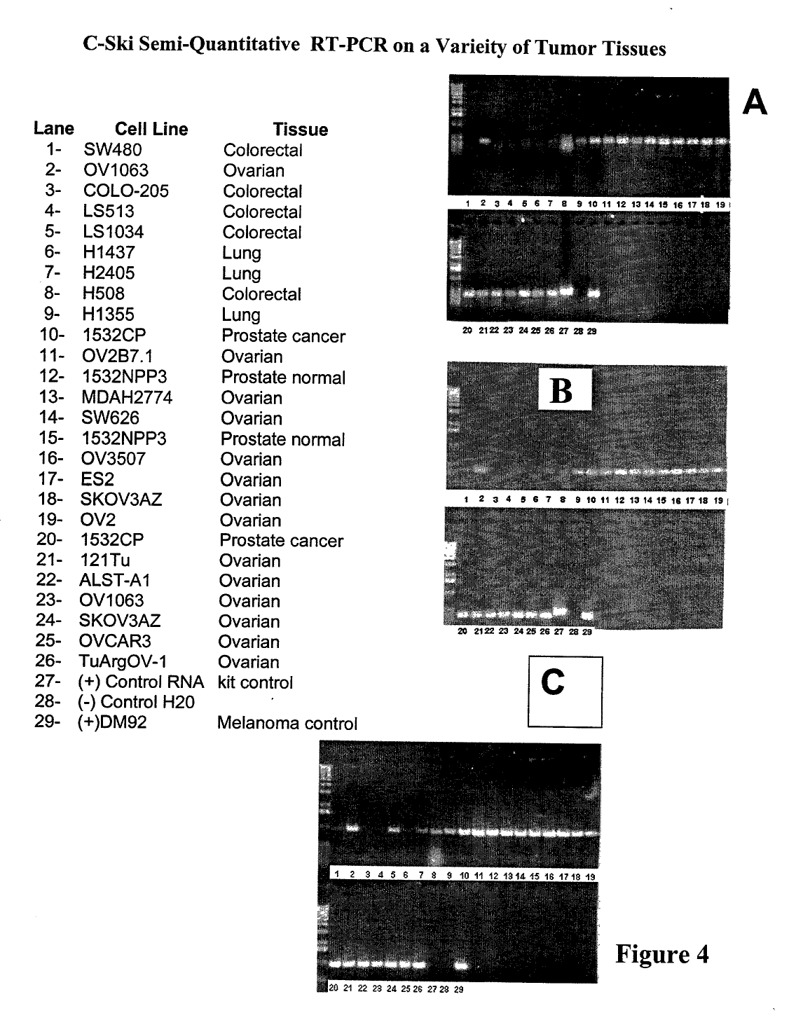

RT-PCR with c-ski-specific primers was also used to assess c-ski mRNA expression in tumor lines of varying origin (FIG. 4). In this analysis, 14 of 14 ovarian carcinoma lines, 2 of 3 lung carcinoma lines, 2 of 2 E6/7 transformed prostate carcinoma, 2 of 2 E6/6 transformed normal prostate expressed c-ski. Analysis of c-ski expression in colorectal carcinoma was more difficult due to overall loss of RT-PCR amplifiable material, however, at least 1 of 5 colorectal carcinoma lines appeared to express the c-ski oncogene at low levels. The fact that the c-ski oncogene is not only expressed in melanoma, but is expressed in a variety of tumor types including ovarian, prostate, lung, and colorectal carcinoma indicates that it will have utility in the treatment and diagnosis of a variety of different cancer types.

### EXAMPLE 8

### Motif-based Search of the c-ski Oncoprotein for HLA-A1, -A2, or -A3 Associated Peptides

The c-ski oncoprotein was scanned for peptides containing a motif that would allow binding to HLA-A1, -A2, or -A3. The search algorithm is located at http://bimas.cit.nih.gov/molbio/hla_bind/. The algorithm ranks peptides, derived from an inputted sequence, by their predicted half-time of dissociation at 37° C. and pH6.5 with the selected class I MHC molecule. The algorithm is based on the assumption that each amino acid in the peptide independently contributes to the binding to the class I MHC molecule. Preferred anchor motif residues have coefficients that are greater than 1, residues that are known to inhibit binding have coefficients that are less than 1, and residues that have no effect on binding have coefficients equal to 1. The coefficients are multiplied together, and ultimately multiplied by a final constant that is specific for each class I MHC molecule. The final number is the predicted half-time of dissociation, and the larger this number is, the more stable the binding is predicted to be. The top ten binding peptides for each of HLA-A1, -A2, and -A3 were selected for further study. These peptides are listed in Table 1 and include SEQ ID NOs:1, 5-33.

TABLE 1
HLA-A2, -A2, and -A3 Motif-predicted Peptides from c-ski*

| HLA-A1 | | | HLA-A2 | | | HLA-A3 | | |
|---|---|---|---|---|---|---|---|---|
| SEQ ID | | | SEQ ID | | | SEQ ID | | |
| NO: | Sequence | Score | NO: | Sequence | Score | NO: | Sequence | Score |
| 5 | SSEPPASIR | 135 | 15 | LMYPPHKFV | 1502.9 | 25 | KLSAALQAK | 90 |
| 6 | TADQLEILK | 50 | 16 | CLPQILNSV | 160 | 26 | KLREATEAK | 90 |
| 7 | ATEPPPVLH | 45 | 17 | SLHQELEFL | 132.4 | 27 | KMRVKQEEK | 60 |
| 8 | ELEFLRVAK | 36 | 18 | RLGRCLDDV | 78.4 | 28 | ALEGGLDTK | 45 |
| 9 | ATEAKRNLR | 22.5 | 19 | YILLSQDYT | 47 | 1 | ALAPPAQQK | 45 |
| 10 | ALEGGLDTK | 18 | 20 | VMGILPFSA | 35.4 | 29 | RVYHECFGK | 30 |
| 11 | RAENEKKMK | 18 | 21 | ELSERSVRV | 21.4 | 30 | LLSQDYTGK | 20 |
| 12 | ELEQARQAR | 9 | 22 | FSLQQINAV | 17.8 | 31 | RLRAENEKK | 20 |
| 13 | EAEVEVESR | 9 | 23 | KLTVDTPGA | 17.4 | 32 | KMKEANESR | 18 |
| 14 | LSERSVRVY | 6.75 | 24 | ALPSAVPDA | 11.4 | 33 | FLHEWKMR | 13.5 |

5*Here, the term ″Score″ refers to the predicted half-time of dissociation as described in Example 8.

### Reference List

Grant, G. A., ed. Synthetic Peptides: A User's Guide, 1992, W. H. Freeman and Company, New York

Coligan, J. E., Kruisbeek, A., Margulies, D. H., Shevach, E. M., and Strober, W., ed. Current Protocols in Immunology, 1999, John Wiley & Sons, Inc., New York

Ausubel, F. M., Brent, R., Kingston, R. E., Moore, D. D., Seidman, J. G., Smith, J. A., and Struhl, K., ed. Current Protocols in Molecular Biology, 1999, John Wiley & Sons, Inc., New York

Coligan, J. E., Dunn, B. M., Ploegh, H. L., Speicher, D. W., and Wingfield, P. T., ed. Current Protocols in Protein Science, 1999, John Wiley & Sons, Inc., New York

Boon, T., Cerottini, J. C., Van den Eynde, B., van der Bruggen, P., and Van Pel, A. (1994) Tumor antigens recognized by T lymphocytes. [Review] [176 refs]. Ann.Rev.Immunol. 12:337-365.

Celluzzi, C. M., Mayordomo, J. I., Storkus, W. J., Lotze, M. T., Falo, L. D., and Jr (1-1-1996) Peptide-pulsed dendritic cells induce antigen-specific CTL-mediated protective tumor immunity [see comments]. J.Exp.Med. 183:283-287.

Colmenares, C. and Stavnezer, E. (1990) Structure and activities of the ski oncogene. Cancer Biology 1:383-387.

Engelhard, V. H. (1994) Structure of peptides associated with class I and class II MHC molecules. [Review] [137 refs]. Ann.Rev.Immunol. 12:181-207.

Fumagalli, S, Doneda, L, Nomura, N., and Larizza, L. (1993) Expression of the c-ski proto-oncogene in human melanoma cell lines. Melanoma Res. 3:23-27.

Fumagalli, S., Doneda, L., Nomura, N., and Larizza, L. (1993) Expression of the c-ski proto-oncogene in human melanoma cell lines. Melanoma Res. 3:23-27.

Gilboa, E. (1999) The makings of a tumor rejection antigen. Immunity 11:263-270.

Harding, C. H. III (1992) Electroporation of exogenous antigen into cytosol for antigen processing and class I major histocompatibility complex (MHC) presentation: weak base amines and hypothermia (18° C.) inhibit the class I MHC processing pathway. Eur.J.Immunol. 22:1865-1869.

Henderson, R. A., Cox, A. L., Sakaguchi, K., Appella, E., Shabanowitz, J., Hunt, D. F., and Engelhard, V. H. (11-1-1993) Direct identification of an endogenous peptide recognized by multiple HLA-A2.1-specific cytotoxic T cells. Proc.Natl.Acad.Sci.U.S.A 90:10275-10279.

Hogan, K. T., Eisinger, D. P., Cupp, S. B., Lekstrom, K. J., Deacon, DD, Shabanowitz, J., Hunt, D. F., Engelhard, V. H., Slingluff, C. L. Jr, and Ross, M. M. (11-15-1998) The peptide recognized by HLA-A68.2-restricted, squamous cell carcinoma of the lung-specific cytotoxic T lymphocytes is derived from a mutated elongation factor 2 gene. Cancer Res. 58:5144-5150.

Hunt, D. F., Henderson, R. A., Shabanowitz, J., Sakaguchi, K., Michel, H., Sevilir, N., Cox, A. L., Appella, E., and Engelhard, V. H. (3-6-1992) Characterization of peptides bound to the class I MHC molecule HLA-A2.1 by mass spectrometry [see comments]. Science 255:1261-1263.

Hunt, D. F., Yates, J. R., Shabanowitz, J., Winston, S., Hauer, and C R (1986) Protein sequencing by tandem mass spectrometry. Proc.Natl.Acad.Sci.U.S.A 83:6233-6237.

Ioannides, C. G., Freedman, R. S., Platsoucas, C. D., Rashed, S., and Kim, Y. P. (3-1-1991) Cytotoxic T cell clones isolated from ovarian tumor-infiltrating lymphocytes recognize multiple antigenic epitopes on autologous tumor cells. J.Immunol. 146:1700-1707.

Jacob, L., Somasundaram, R., Smith, W., Monos, D., Basak, S., Marincola, F., Pereira, S, and Herlyn, D. (1997) Cytotoxic T-cell clone against rectal carcinoma induced by stimulation of a patient's peripheral blood mononuclear cells with autologous cultured tumor cells. Int.J.Cancer 71:325-332.

Ljunggren, H.-G., Stam, N. J., Ohlen, C., Neefjes, J. J., Hoglund, P., Heemels, M.-T., Bastin, J., Schumacher, T. N. M., Townsend, A., Karre, K., and Ploegh, H. L. (1990) Empty MHC class I molecules come out in the cold. Nature 346:476-480.

Mayordomo, J. I., Zorina, T., Storkus, W. J., Zitvogel, L., Celluzzi, Falo, L. D., Melief, C. J., Ildstad, S. T., Kast, W. M., DeLeo, A. B., and Lotze, M. T. (1995) Bone marrow-derived dendritic cells pulsed with synthetic tumour peptides elicit protective and therapeutic antitumour immunity. Nat.Med. 1:1297-1302.

Moore, M. W., Carbone, F. R., and Bevan, M. J. (1988) Introduction of soluble protein into the class I pathway of antigen processing and presentation. Cell 54:777-785.

North, R. J., LaCourse, R., and Ryan, L. (1999) Vaccinated mice remain more susceptible to Mycobacterium tuberculosis infection initiated via the respiratory route than via the intravenous route. Infect.Immun. 67:2010-2012.

Parkhurst, M. R., Salgaller, M. L., Southwood, S., Robbins, P. F., Sette, A., Rosenberg, S. A., and Kawakami, Y. (9-15-1996) Improved induction of melanoma-reactive CTL with peptides from the melanoma antigen gp100 modified at HLA-A*0201-binding residues. J.Immunol. 157:2539-2548.

Peiper, M., Goedegebuure, P. S., Linehan, D. C., Ganguly, E., Douville, C. C., and Eberlein, T. J. (1997) The HER2/neu-derived peptide p654-662 is a tumor-associated antigen in human pancreatic cancer recognized by cytotoxic T lymphocytes. Eur.J.Immunol. 27:1115-1123.

Peoples, G. E., Schoof, D. D., Andrews, J. V., Goedegebuure, P. S., and Eberlein, T. J. (1993) T-cell recognition of ovarian cancer. Surgery 114:227-234.

Perez-Diez, A., Butterfield, L. H., Li, L., Chakraborty, N. G., Economou, J. S., and Mukherji, B. (1998) Generation of CD8+ and CD4+ T-cell response to dendritic cells genetically engineered to express the MART-1/Melan-A gene. Cancer Res. 58:5305-5309.

Posnett, D. N., McGrath, H., and Tam, J. P. (1988) A novel method for producing anti-peptide antibodies. Production of site-specific antibodies to the T cell antigen receptor beta-chain. J.Biol.Chem. 263:1719-1725.

Reddy, R., Zhou, F., Huang, L., Carbone, F., Bevan, M., and Rouse, B. T. (1991) pH sensitive liposomes provide an efficient means of sensitizing target cells to class I restricted CTL recognition of a soluble protein. J.Immunol.Methods 141:157-163.

Riddell, S. R. and Greenberg, P. D. (4-17-1990) The use of anti-CD3 and anti-CD28 monoclonal antibodies to clone and expand human antigen-specific T cells. J.Immunol.Methods 128:189-201.

Riddell, S. R., Watanabe, K. S., Goodrich, J. M., Li, C. R., Agha, M. E., and Greenberg, P. D. (7-10-1992) Restoration of viral immunity in immunodeficient humans by the adoptive transfer of T cell clones [see comments]. Science 257:238-241.

Rock, K. L. and Golde, U. (1999) Degradation of cell proteins and the generation of MHC class I-presented peptides. Ann.Rev.Immunol. 17:739-779.

Rosenberg, S. A. (1999) A new era for cancer immunotherapy based on the genes that encode cancer antigens. [Review] [79 refs]. Immunity 10:281-287.

Rosenberg, S. A., Packard, B. S., Aebersold, P. M., Solomon, D., Topalian, S. L., Toy, S. T., Simon, P., Lotze, M. T., Yang, J. C., and Seipp, C. A. (12-22-1988) Use of tumor-infiltrating lymphocytes and interleukin-2 in the immunotherapy of patients with metastatic melanoma. A preliminary report [see comments]. N.Engl.J.Med. 319:1676-1680.

Rosenberg, S. A., Yang, J. C., Schwartzentruber, D. J., Hwu, P., Marincola, F. M., Topalian, S. L., Restifo, N. P., Dudley, M. E., Schwarz, S. L., Spiess, P. J., Wunderlich, J. R., Parkhurst, M. R., Kawakami, Y., Seipp, C. A., Einhorn, J. H., and White, D. E. (1998). Immunologic and therapeutic evaluation of a synthetic peptide vaccine for the treatment of patients with metastatic melanoma. Nat.Med. 4:321-327.

Molecular Cloning: A Laboratory Manual, 1989, Cold Spring Harbor Laboratory Press, Cold Spring Harbor

Schendel, D. J., Gansbacher, B., Oberneder, R., Kriegmair, M., Hofstetter, A., Riethmuller, G., and Segurado, O. G. (10-15-1993) Tumor-specific lysis of human renal cell carcinomas by tumor-infiltrating lymphocytes. I. HLA-A2-restricted recognition of autologous and allogeneic tumor lines. J.Immunol. 151:4209-4220.

Skipper, J. C., Kittlesen, D. J., Hendrickson, R. C., Deacon, D. D., Harthun, N. L., Wagner, SN, Hunt, D. F., Engelhard, V. H., Slingluff, C. L., and Jr (12-1-1996) Shared epitopes for HLA-A3-restricted melanoma-reactive human CTL include a naturally processed epitope from Pmel-17/gp100. J.Immunol. 157:5027-5033.

Slingluff, C. L. Jr, Cox, A. L., Henderson, R. A., Hunt, D. F., and Engelhard, V. H. (4-1-1993) Recognition of human melanoma cells by HLA-A2.1-restricted cytotoxic T lymphocytes is mediated by at least six shared peptide epitopes. J.Immunol. 150:2955-2963.

Slingluff, C. L. Jr, Cox, A. L., Stover, J. M. Jr, Moore, M. M., Hunt, D F, and Engelhard, V. H. (5-15-1994) Cytotoxic T-lymphocyte response to autologous human squamous cell cancer of the lung: epitope reconstitution with peptides extracted from HLA-Aw68. Cancer Res. 54:2731-2737.

Slovin, S. F., Lackman, R. D., Ferrone, S., Kiely, P. E., and Mastrangelo, M. J. (1987) Cellular immune response to human sarcomas: cytotoxic T cell clones reactive with autologous sarcomas. I. Development, phenotypes, and specificity. J.Immunol. 137:3042-3048.

Tuting, T., Wilson, C. C., Martin, D. M., Kasamon, Y. L., Rowles, J., Ma, D. I., Slingluff, Jr, C. L., Wagner, S. N., van der Bruggen, P., Baar, J., Lotze, M. T., and Storkus, W. J. (2-1-1998) Autologous human monocyte-derived dendritic cells genetically modified to express melanoma antigens elicit primary cytotoxic T cell responses in vitro: enhancement by cotransfection of genes encoding the Th1-biasing cytokines IL-12 and IFN-alpha. J.Immunol. 160:1139-1147.

Vogel, G. (1999) A new blocker for the TGF-pathway. Science 286:665-665.

Walter, E. A., Greenberg, P. D., Gilbert, M. J., Finch, R. J., Watanabe, KS, Thomas, E. D., and Riddell, S. R. (1995) Reconstitution of cellular immunity against cytomegalovirus in recipients of allogeneic bone marrow by transfer of T-cell clones from the donor [see comments]. N.Engl.J.Med. 333:1038-1044.

Watts, C. (1997) Capture and processing of exogenous antigens for presentation on MHC molecules. Ann.Rev.Immunol. 15:821-850.

Wolfel, T., Herr, W., Coulie, P., Schmitt, U., Meyer, Zum Buschenfelde K H, and Knuth, A. (1993) Lysis of human pancreatic adenocarcinoma cells by autologous HLA-class I-restricted cytolytic T-lymphocyte (CTL) clones. Int.J.Cancer 54:636-644.

Yasumura, S., Hirabayashi, H., Schwartz, D. R., Toso, J. F., Johnson, J. T., Herberman, R B, and Whiteside, T. L. (3-15-1993) Human cytotoxic T-cell lines with restricted specificity for squamous cell carcinoma of the head and neck. Cancer Res. 53:1461-1468.

Yewdell, J. W. and Bennink, J. R. (1999) Immunodominance in major histocompatibility complex class I-restricted T lymphoycte responses. Ann.Rev.Immunol. 17:51-88.

Yoshino, I., Goedegebuure, P. S., Peoples, G. E., Parikh, A. S., DiMaio, J. M., Lyerly, H. K., Gazdar, A. F., and Eberlein, T. J. (7-1-1994) HER2/neu-derived peptides are shared antigens among human non-small cell lung cancer and ovarian cancer. Cancer Res. 54:3387-3390.

Zeh, H. J., III, Leder, G. H., Lotze, M. T., Salter, R. D., Tector, M., Stuber, G., Modrow, S, and Storkus, W. J. (1994) Flow-cytometric determination of peptide-class I complex formation. Identification of p53 peptides that bind to HLA-A2. Hum.Immunol. 39:79-86.

Zitvogel, L., Mayordomo, J. I., Tjandrawan, T., DeLeo, A. B., Clarke, M. R., Lotze, M. T., and Storkus, W. J. (1-1-1996) Therapy of murine tumors with tumor peptide-pulsed dendritic cells: dependence on T cells, B7 costimulation, and T helper cell 1-associated cytokines [see comments]. J.Exp.Med. 183:87-97.

What is claimed is:

1. An isolated peptide of at least 8 amino acid residues in length and having an amino acid sequence at least 85% identical to the sequence selected from the group consisting of SEQ ID NOS: 1, 4 to 33.

2. An isolated peptide of no more than about 14 amino acids in length comprising a sequence selected from the group consisting of SEQ ID NOS: 1, 4 to 33.

3. An isolated nonapeptide having an amino acid sequence selected from the group consisting of SEQ ID NOS: 1, 4 to 33.

4. An isolated nonapeptide having a sequence differing by no more than 1 amino acid from a sequence selected from the group consisting of SEQ ID NOS: 1, 4 to 33.

5. The isolated nonapeptide of claim 4 wherein the amino acid difference is a substitution of one conservative amino acid for another.

6. An immunogen comprising one or more isolated peptides selected from the group consisting of the peptides of claims 1, 2, 3, 4 and 5.

7. An immunogen comprising a polypeptide of the amino acid sequence of SEQ ID NO:2.

8. An immunogen comprising a polypeptide, wherein said polypeptide comprises one or more epitopes selected from the peptides of claims 1 through 5, and wherein each said epitopic peptide may be present in one or more copies.

9. An immunogen comprising a polynucleotide sequence coding for at least one cytotoxic T lymphocyte (CTL) epitope, wherein said epitope is selected from a group consisting of the peptides of claims 1, 2, 3, 4, and 5 and operably linked to a promoter, and wherein the polynucleotide sequence does not include the entire c-ski gene of SEQ ID NO:3.

10. A vaccine composition comprising an immunologically active amount of the immunogens of claims 6, 7, 8, and 9.

11. A method for inducing a CTL response in vitro that is specific for a tumor cell expressing at least one of HLA-A1, -2, or -A3 and the c-ski oncogene (SEQ ID NO:3), whereby the method comprises contacting a precursor CTL with an immunogenic peptide selected from a group comprising the peptide immunogens of claims 6 and 7 under conditions that generate a CTL response to the tumor cell.

12. A method of inducing a CTL response in vitro that is specific for a tumor cell expressing at least one of HLA-A1, -A2, or -A3, and the c-ski oncogene (SEQ ID NO:3), whereby the method comprises contacting a precursor CTL with an antigen presenting cell that has exogenously acquired the immunogen of claim 8.

13. A method of inducing a CTL response in vitro that is specific for a tumor cell expressing at least one of HLA-A1, -A2,or -A3, and the c-ski oncogene (SEQ ID NO:3), whereby the method comprises contacting a precursor CTL with an antigen presenting cell that is expressing a polynucleotide of claim 10.

14. A method of treating a subject with cancer characterized by tumor cells expressing at least one of HLA-A1, -A2, or -A3, and the c-ski oncogene(SEQ ID NO:3), whereby the CTLs induced by the methods of claims 11, 12, or 13 are administered in an amount sufficient to destroy the tumor cells through direct lysis or to effect the destruction of the tumor cells indirectly through the elaboration of cytokines.

15. A method of treating a subject with cancer characterized by tumor cells expressing any class I MHC molecule and a gene of SEQ ID NO:3, whereby the CTLs of claim 14 are administered in an amount sufficient to destroy the tumor cells through direct lysis or to effect the destruction of the tumor cells indirectly through the elaboration of cytokines.

16. The method of claims 14 and 15 wherein said cancer is melanoma.

17. The method of claims 14 and 15 wherein said cancer is colorectal carcinoma.

18. The method of claims 14 and 15 wherein said cancer is ovarian carcinoma.

19. The method of claims 14 and 15 wherein said cancer is lung carcinoma.

20. The method of claims 14 and 15 wherein said cancer is prostate carcinoma.

21. A method for inducing a CTL response in a subject, whereby the method comprises administering at least one CTL epitope, wherein said epitope is selected from a group consisting of the immunogens of claims 6, 8, 9 10, and 11 to an HLA-A1, -A2, or -A3 positive subject, in an amount sufficient to induce a CTL response to tumor cells expressing at least one of HLA-A1, -A2, and -A3, and the c-ski oncogene (SEQ ID NO:3).

22. A method for inducing a CTL response in a subject, whereby the method comprises administering the immunogen of claim 9 to a subject, in an amount sufficient to induce a CTL response to tumor cells expressing class I MHC molecules and the c-ski oncogene (SEQ ID NO:3).
